(19) United States
(12) Patent Application Publication
(10) Pub. No.: US 2007/0276439 A1
(43) Pub. Date: Nov. 29, 2007

(54) COLLECTING SLEEP QUALITY INFORMATION VIA A MEDICAL DEVICE

(75) Inventors: Keith A. Miesel, St. Paul, MN (US); Kenneth T. Heruth, Edina, MN (US); Jonathan C. Werder, Corcoran, MN (US); Nina M. Graves, Minnetonka, MN (US); Steve R. LaPorte, San Antonio, TX (US)

Correspondence Address: SHUMAKER & SIEFFERT, P. A., 1625 RADIO DRIVE, SUITE 300, WOODBURY, MN, 55125, US

(73) Assignee: Medtronic, Inc.

(21) Appl. No.: 11/691,376

(22) Filed: Mar. 26, 2007

Related U.S. Application Data
(63) Continuation-in-part of application No. 11/081,811, filed on Mar. 16, 2005.
Application No. 11/081,811 is a continuation-in-part of application No. 10/826,925, filed on Apr. 15, 2004.
(60) Provisional application No. 60/553,783, filed on Mar. 16, 2004.
(60) Provisional application No. 60/785,678, filed on Mar. 24, 2006.

(51) Int. Cl. A61N 1/00

(52) U.S. Cl. 607/2

(57) ABSTRACT

At least one of a medical device, such as an implantable medical device, a monitor, and a computing device determines values for one or more metrics that indicate the quality of a patient's sleep. Sleep efficiency, sleep latency, and time spent in deeper sleep states are example sleep quality metrics for which values may be determined. In some embodiments, determined sleep quality metric values are associated with a current therapy parameter set. In some embodiments, a programming device presents sleep quality information to a user based on determined sleep quality metric values. A clinician may use the sleep quality information presented by the programming device to evaluate the effectiveness of therapy delivered to the patient by the medical device, to adjust the therapy delivered by the medical device, or to prescribe a therapy not delivered by the medical device in order to improve the quality of the patient's sleep.

This application is a continuation-in-part of U.S. application Ser. No. 11/081,811, filed Mar. 16, 2005, which is a continuation-in-part of U.S. application Ser. No. 10/826,925, filed Apr. 15, 2004, which claims the benefit of U.S. provisional application No. 60/553,783, filed Mar. 16, 2004. This application also claims the benefit of U.S. provisional application No. 60/785,678, filed Mar. 24, 2006. The entire content of each of these applications is incorporated herein by reference.

### TECHNICAL FIELD

The invention relates to medical devices and, more particularly, to medical devices that monitor physiological parameters.

### BACKGROUND

In some cases, an ailment that a patient has may affect the quality of the patient's sleep. For example, chronic pain may cause a patient to have difficulty falling asleep, and may disturb the patient's sleep, e.g., cause the patient to wake. Further, chronic pain may cause the patient to have difficulty achieving deeper sleep states, such as one or more of the nonrapid eye movement (NREM) sleep states.

Other ailments that may negatively affect patient sleep quality include movement disorders, such as tremor, Parkinson's disease, multiple sclerosis, or spasticity. The uncontrolled movements associated with such movement disorders may cause a patient to have difficulty falling asleep, disturb the patient's sleep, or cause the patient to have difficulty achieving deeper sleep states. Movement disorders may include tremor, Parkinson's disease, multiple sclerosis, epilepsy, or spasticity, as well disorders including sleep apnea, congestive heart failure, gastrointestinal disorders and incontinence may negatively affect patient sleep quality. Psychological disorders, such as depression, mania, bipolar disorder, or obsessive-compulsive disorder, sleep apnea, congestive heart failure, gastrointestinal disorders and incontinence may also similarly affect the ability of a patient to sleep, or at least experience quality sleep. In the case of depression, a patient may “sleep” for long periods of the day, but the sleep is not restful, e.g., includes excessive disturbances and does not include deeper, more restful sleep states. Any of a variety of neurological disorders, including movement disorders, psychological disorders and chronic pain, may negatively effect sleep quality. In some cases, these ailments are treated via an implantable medical device (IMD), such as an implantable stimulator or drug delivery device.

Further, in some cases, poor sleep quality may increase the symptoms experienced by a patient due to an ailment. For example, poor sleep quality has been linked to increased pain symptoms in chronic pain patients, and may also result in increased movement disorder symptoms in movement disorder patients. Further, poor sleep quality may exacerbate many psychological disorders, such as depression. The link between poor sleep quality and increased symptoms is not limited to ailments that negatively impact sleep quality, such as those listed above. Nonetheless, the condition of a patient with such an ailment may progressively worsen when symptoms disturb sleep quality, which in turn increases the frequency and/or intensity of symptoms.

### SUMMARY

In general, the invention is directed to techniques for collecting information that relates to the quality of patient sleep via a medical device, such as an implantable medical device (IMD). In particular, values for one or more metrics that indicate the quality of the patient's sleep are determined based on physiological parameters monitored by a medical device. In some embodiments, sleep quality information is presented to a user based on the sleep quality metric values. A clinician, for example, may use the presented sleep quality information to evaluate the effectiveness of therapy delivered to the patient by the medical device, to adjust the therapy delivered by the medical device, or to prescribe a therapy not delivered by the medical device in order to improve the quality of the patient's sleep.

The medical device that delivers the therapy or a separate monitoring device monitors one or more physiological parameters of the patient. Example physiological parameters that the medical device may monitor include activity level, posture, heart rate, electrocardiogram (ECG) morphology, respiration rate, respiratory volume, blood pressure, blood oxygen saturation, partial pressure of oxygen within blood, partial pressure of oxygen within cerebrospinal fluid, muscular activity and tone, core temperature, subcutaneous temperature, arterial blood flow, melatonin level within one or more bodily fluids, brain electrical activity, eye motion, and galvanic skin response. In order to monitor one or more of these parameters, the medical device or monitoring device may include, or be coupled to one or more sensors, each of which generates a signal as a function of one or more of these physiological parameters.

The medical device or monitoring device may determine a value of one or more sleep quality metrics based on the one or more monitored physiological parameters, and/or the variability of one or more of the monitored physiological parameters. In other embodiments, one or both of the medical device or monitoring device records values of the one or more physiological parameters, and provides the physiological parameter values to a programming device, such as a clinician programming device or a patient programming device, or another computing device. In such embodiments, the programming or other computing device determines values of one or more sleep quality metrics based on the physiological parameter values received from the medical device and/or the variability of one or more of the physiological parameters. The medical device or monitoring device may provide the recorded physiological parameter values to the programming or other computing device in real time, or may provide physiological parameter values recorded over a period of time to the programming or other computing device when interrogated.

Sleep efficiency and sleep latency are example sleep quality metrics for which a medical device or programming device may determine values. Sleep efficiency may be measured as the percentage of time while the patient is attempting to sleep that the patient is actually asleep. Sleep latency may be measured as the amount of time between a first time when the patient begins attempting to fall asleep and a second time when the patient falls asleep, and thereby indicates how long a patient requires to fall asleep.

The time when the patient begins attempting to fall asleep may be determined in a variety of ways. For example, the patient may provide an indication that he or she is trying to fall asleep, e.g., via a patient programming device. In other embodiments, the medical device or monitoring may monitor the activity level of the patient, and the time when the patient is attempting to fall asleep may be identified by determining whether the patient has remained inactive for a threshold period of time, and identifying the time at which the patient became inactive. In still other embodiments, the medical device or monitoring device may monitor patient posture, and the medical device or a programming device may identify the time when the patient is recumbent, e.g., lying down, as the time when the patient is attempting to fall asleep. In these embodiments, the medical device or monitoring device may also monitor patient activity, and either the medical device, monitoring device, programming device, or other computing device may confirm that the patient is attempting to sleep based on the patient's activity level.

As another example, the medical device or monitoring device may determine the time at which the patient begins attempting to fall asleep based on the level of melatonin within one or more bodily fluids, such as the patient's blood, cerebrospinal fluid (CSF), or interstitial fluid. The medical device or monitoring device may also determine a melatonin level based on metabolites of melatonin located in the saliva or urine of the patient. Melatonin is a hormone secreted by the pineal gland into the bloodstream and the CSF as a function of exposure of the optic nerve to light, which synchronizes the patient's circadian rhythm. In particular, increased levels of melatonin during evening hours may cause physiological changes in the patient, which, in turn, may cause the patient to attempt to fall asleep. The medical device or monitoring device may, for example, detect an increase in the level of melatonin, and estimate the time that the patient will attempt to fall asleep based on the detection.

The time at which the patient has fallen asleep may be determined based on the activity level of the patient and/or one or more of the other physiological parameters that may be monitored by the medical device as indicated above. For example, a discernable change, e.g., a decrease, in one or more physiological parameters, or the variability of one or more physiological parameters, may indicate that the patient has fallen asleep. In some embodiments, a sleep probability metric value may be determined based on a value of a physiological parameter monitored by the medical device. In such embodiments, the sleep probability metric value may be compared to a threshold to identify when the patient has fallen asleep. In some embodiments, a plurality of sleep probability metric values are determined based on a value of each of a plurality of physiological parameters, the sleep probability values are averaged or otherwise combined to provide an overall sleep probability metric value, and the overall sleep probability metric value is compared to a threshold to identify the time that the patient falls asleep.

Other sleep quality metrics that may be determined include total time sleeping per day, the amount or percentage of time sleeping during nighttime or daytime hours per day, and the number of apnea and/or arousal events per night. In some embodiments, in which sleep state the patient currently is, e.g., rapid eye movement (REM), or one of the nonrapid eye movement (NREM) states (S1, S2, S3, S4) may be determined based on physiological parameters monitored by the medical device. The amount of time per day spent in these various sleep states may be a sleep quality metric. Because they provide the most “refreshing” type of sleep, the amount of time spent in one or both of the S3 and S4 sleep states, in particular, may be determined as a sleep quality metric. In some embodiments, average or median values of one or more sleep quality metrics over greater periods of time, e.g., a week or a month, may be determined as the value of the sleep quality metric. Further, in embodiments in which values for a plurality of the sleep quality metrics are determined, a value for an overall sleep quality metric may be determined based on the values for the plurality of individual sleep quality metrics.

In some embodiments, the medical device delivers a therapy. At any given time, the medical device delivers the therapy according to a current set of therapy parameters. For example, in embodiments in which the medical device is a neurostimulator, a therapy parameter set may include a pulse amplitude, a pulse width, a pulse rate, a duty cycle, and an indication of active electrodes. Different therapy parameter sets may be selected, e.g., by the patient via a programming device or a the medical device according to a schedule, and parameters of one or more therapy parameter sets may be adjusted by the patient to create new therapy parameter sets. In other words, over time, the medical device delivers the therapy according to a plurality of therapy parameter sets.

The therapy may be directed to treating any number of disorders. For example, the therapy may be directed to treating a non-respiratory neurological disorder, such as a movement disorder or psychological disorder. Example movement disorders for which therapy may be provided are Parkinson's disease, essential tremor and epilepsy. Non-respiratory neurological disorders do not include respiratory disorders, such as sleep apnea.

In embodiments in which the medical device determines sleep quality metric values, the medical device may identify the current therapy parameter set when a value of one or more sleep quality metrics is collected, and may associate that value with the therapy parameter set. For example, for each available therapy parameter set the medical device may store a representative value of each of one or more sleep quality metrics in a memory with an indication of the therapy programs with which that representative value is associated. A representative value of sleep quality metric for a therapy parameter set may be the mean or median of collected sleep quality metric values that have been associated with that therapy parameter set. In other embodiments in which a programming device or other computing device determines sleep quality metric values, the medical device may associate recorded physiological parameter values with the current therapy parameter set in the memory. Further, in embodiments in which a separate monitoring device records physiological parameter values or determines sleep quality metric values, the monitoring device may mark recorded physiological parameter values or sleep quality metric values with a current time in a memory, and the medical device may store an indication of a current therapy parameter set and time in a memory. A programming device of other computing device may receive indications of the physiological parameter values or sleep quality metrics and associated times from the monitoring device, and indications of the therapy parameter sets and associated times from the medical device, and may associate the physiological parameter values or sleep quality metrics with the therapy parameter set that was delivered by the medical device when the physiological parameter values or sleep quality metrics were collected.

A programming device or other computing device according to the invention may be capable of wireless communication with the medical device, and may receive sleep quality metric values or recorded physiological parameter values from the medical device or a separate monitoring device. In either case, when the computing device either receives or determines sleep quality metric values, the computing device may provide sleep quality information to a user based on the sleep quality metric values. For example, the computing device may be a patient programmer, and may provide a message to the patient related to sleep quality. The patient programmer may, for example, suggest that the patient visit a clinician for prescription of sleep medication or for an adjustment to the therapy delivered by the medical device. As other examples, the patient programmer may suggest that the patient increase the intensity of therapy delivered by the medical device during nighttime hours relative to previous nights, or select a different therapy parameter set for use during sleep than the patient had selected during previous nights. Further, the patient programmer may provide a message that indicates the quality of sleep to the patient to, for example, provide the patient with an objective indication of whether his or her sleep quality is good, adequate, or poor.

In other embodiments, the computing device is a clinician programmer that presents information relating to the quality of the patient's sleep to a clinician. The clinician programmer may present, for example, a trend diagram of values of one or more sleep quality metrics over time. As other examples, the clinician programmer may present a histogram or pie chart illustrating percentages of time that a sleep quality metric was within various value ranges.

As indicated above, the computing device may receive representative values for one or more sleep quality metrics or the physiological parameter values from the therapy delivering medical device or separate monitoring device. The computing device may receive information identifying the therapy parameter set with which the representative values are associated, or may itself associate received physiological parameter or sleep quality metric values with therapy parameter sets based on time information received from one or more devices. In embodiments in which the computing device receives physiological parameter values, the computing device may determine sleep quality metric values associated with the plurality of parameter sets based on the physiological parameter values, and representative sleep quality metric values for each of the therapy parameter sets based on the sleep quality metric values associated with the therapy parameter sets. In some embodiments, the computing device may determine the variability of one or more of the physiological parameters based on the physiological parameter values received from the medical device or monitoring device, and may determine sleep quality metric values based on the physiological parameter variabilities.

The computing device may display a list of the therapy parameter sets to the clinician ordered according to their associated representative sleep quality metric values. Such a list may be used by the clinician to identify effective or ineffective therapy parameter sets. Where a plurality of sleep quality metric values are determined, the programming device may order the list according to values of a user-selected one of the sleep quality metrics.

In other embodiments, a system according to the invention does not include a programming or other computing device. For example, an external medical device according to the invention may include a display, determine sleep quality metric values, and display sleep quality information to a user via the display based on the sleep quality metric values.

In one embodiment, the invention is directed to a method that includes monitoring at least one physiological parameter of a patient, determining a value of a metric that is indicative of sleep quality based on the at least one physiological parameter, identifying a current therapy parameter used by a medical device to deliver a therapy to a patient, wherein the therapy comprises at least one of a movement disorder therapy, psychological disorder therapy, or deep brain stimulation, and associating the sleep quality metric value with the current therapy parameter set.

In another embodiment, the invention is directed to a medical system that includes a medical device that delivers at least one of a movement disorder therapy, psychological disorder therapy, or deep brain stimulation to a patient and a monitor that monitors at least one physiological parameter of the patient based on a signal received from at least one sensor. The medical system also includes a processor that determines a value of a metric that is indicative of sleep quality based on the at least one physiological parameter, identifies a current therapy parameter set used by the medical device to deliver the therapy to the patient, and associates the sleep quality metric value with the current therapy parameter set.

In an additional embodiment, the invention is directed to a computer-readable medium including instructions that cause a processor to monitor at least one physiological parameter of a patient and determine a value of a metric that is indicative of sleep quality based on the at least one physiological parameter. The computer-readable medium also includes instructions that cause the processor to identify a current therapy parameter used by a medical device to deliver a therapy to a patient, wherein the therapy comprises at least one of a movement disorder therapy, psychological disorder therapy, or deep brain stimulation and associate the sleep quality metric value with the current therapy parameter set.

Embodiments of the invention may be capable of providing one or more advantages. For example, by providing information related to the quality of a patient's sleep to a clinician and/or the patient, a system according to the invention can improve the course of treatment of an ailment of the patient, such as chronic pain, a movement disorder, or a psychological disorder. Using the sleep quality information provided by the system, the clinician and/or patient can, for example, make changes to the therapy provided by a medical device in order to better address symptoms which are disturbing the patient's sleep. Further, a clinician may choose to prescribe a therapy that will improve the patient's sleep, such as a sleep inducing medication, in situations where poor sleep quality is increasing symptoms experienced by the patient.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIGS. 1A and 1B are conceptual diagrams illustrating example systems that include an implantable medical device that collects sleep quality information according to the invention.

FIGS. 2A and 2B are block diagrams further illustrating the example systems and implantable medical devices of FIGS. 1A and 1B.

FIG. 3 is a logic diagram illustrating an example circuit that detects the sleep state of a patient from the electroencephalogram (EEG) signal.

FIG. 4 is a block diagram illustrating an example memory of the implantable medical device of FIG. 1.

FIG. 5 is a flow diagram illustrating an example method for collecting sleep quality information that may be employed by an implantable medical device.

FIG. 6 is a flow diagram illustrating an example method for associating sleep quality information with therapy parameter sets that may be employed by an implantable medical device.

FIG. 7 is a block diagram illustrating an example clinician programmer.

FIG. 8 is a flow diagram illustrating an example method for presenting sleep quality information to a clinician that may be employed by a clinician programmer.

FIG. 9 illustrates an example list of therapy parameter sets and associated sleep quality information that may be presented by a clinician programmer.

FIG. 10 is a flow diagram illustrating an example method for displaying a list of therapy parameter sets and associated sleep quality information that may be employed by a clinician programmer.

FIG. 11 is a block diagram illustrating an example patient programmer.

FIG. 12 is a flow diagram illustrating an example method for presenting a sleep quality message to a patient that may be employed by a patient programmer.

FIG. 13 is a conceptual diagram illustrating a monitor that monitors values of one or more physiological parameters of the patient instead of, or in addition to, a therapy delivering medical device.

FIG. 14 is a conceptual diagram illustrating a monitor that monitors signals generated by one or more accelerometers disposed on the patient.

FIG. 15 is a flow diagram illustrating an example technique for monitoring the heart rate and breathing rate of a patient by measuring cerebral spinal fluid pressure.

### DETAILED DESCRIPTION

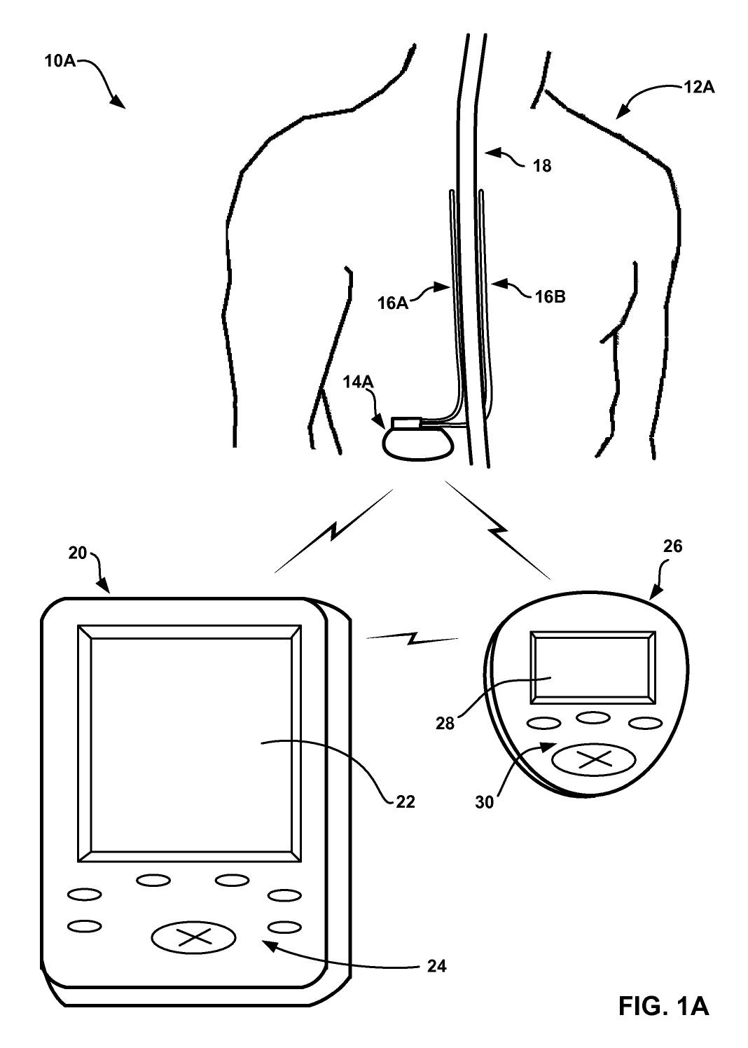

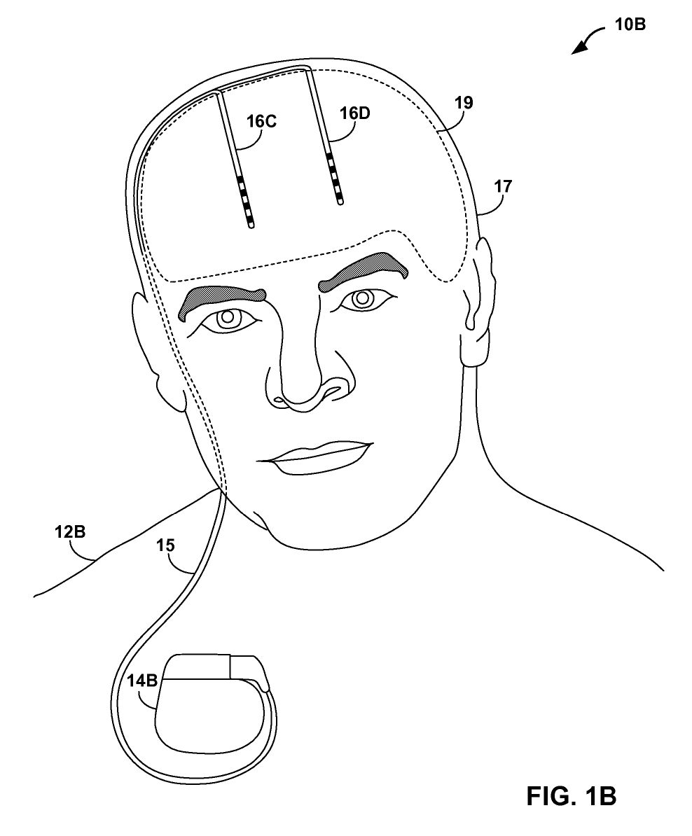

FIGS. 1A and 1B are conceptual diagrams illustrating example systems 10A and 10B (collectively “systems 10”) that respectively include an implantable medical device (IMD) 14A or 14B (collectively “IMDs 14”) that collect information relating to the quality of sleep experienced by a respective one of patients 12A and 12B (collectively “patients 12”) according to the invention. Sleep quality information collected by IMDs 14 may be provided to one or more users, such as a clinician or the patient. Using the sleep quality information collected by IMDs 14, a current course of therapy for one or more ailments of patients 12 may be evaluated, and an improved course of therapy for the ailments may be identified.

In the illustrated example systems 10, IMDs 14 take the form of implantable neurostimulators that deliver neurostimulation therapy in the form of electrical pulses to patients 12. However, the invention is not limited to implementation via implantable neurostimulators. For example, in some embodiments of the invention, an implantable pump or implantable cardiac rhythm management device, such as a pacemaker, may collect sleep quality information. Further, the invention is not limited to implementation via an IMD. In other words, any implantable or external medical device may collect sleep quality information according to the invention.

In the examples of FIGS. 1A and 1B, IMDs 14A and 14B deliver neurostimulation therapy to patients 12A and 12B via leads 16A and 16B, and leads 16C and 16D (collectively “leads 16”), respectively. Leads 16A and 16B may, as shown in FIG. 1A, be implanted proximate to the spinal cord 18 of patient 12A, and IMD 14A may deliver spinal cord stimulation (SCS) therapy to patient 12A in order to, for example, reduce pain experienced by patient 12A. However, the invention is not limited to the configuration of leads 16A and 16B shown in FIG. 1A or the delivery of SCS or other pain therapies.

For example, in another embodiment, illustrated in FIG. 1B, leads 16C and 16D may extend to brain 19 of patient 12B, e.g., through cranium 17 of patient. IMD 14B may deliver deep brain stimulation (DBS) or cortical stimulation therapy to patient 12 to treat any of a variety of non-respiratory neurological disorders, such as movement disorders or psychological disorders. Example therapies may treat tremor, Parkinson's disease, spasticity, epilepsy, depression or obsessive-compulsive disorder. As illustrated in FIG. 1B, leads 16C and 16D may be coupled to IMD 14B via one or more lead extensions 15.

As further examples, one or more leads 16 may be implanted proximate to the pelvic nerves (not shown) or stomach (not shown), and an IMD 14 may deliver neurostimulation therapy to treat incontinence or gastroparesis. Additionally, leads 16 may be implanted on or within the heart to treat any of a variety of cardiac disorders, such as congestive heart failure or arrhythmia, or may be implanted proximate to any peripheral nerves to treat any of a variety of disorders, such as peripheral neuropathy or other types of chronic pain.

The illustrated numbers and locations of leads 16 are merely examples. Embodiments of the invention may include any number of lead implanted at any of a variety of locations within a patient. Furthermore, the illustrated number and location of IMDs 14 are merely examples. IMDs 14 may be located anywhere within patient according to various embodiments of the invention. For example, in some embodiments, an IMD 14 may be implanted on or within cranium 17 for delivery of therapy to brain 19, or other structure of the head of the patient 12.

IMDs 14 delivers therapy according to a set of therapy parameters, i.e., a set of values for a number of parameters that define the therapy delivered according to that therapy parameter set. In embodiments where IMDs 14 delivers neurostimulation therapy in the form of electrical pulses, the parameters in each parameter set may include voltage or current pulse amplitudes, pulse widths, pulse rates, and the like. Further, each of leads 16 includes electrodes (not shown in FIG. 1), and a therapy parameter set may include information identifying which electrodes have been selected for delivery of pulses, and the polarities of the selected electrodes. In embodiments in which IMDs 14 deliver other types of therapies, therapy parameter sets may include other therapy parameters such as drug concentration and drug flow rate in the case of drug delivery therapy. Therapy parameter sets used by IMDs 14 may include a number of parameter sets programmed by one or more clinicians (not shown), and parameter sets representing adjustments made by patients 12 to these preprogrammed sets.

Each of systems 10 may also include a clinician programmer 20 (illustrated as part of system 10A in FIG. 1A). A clinician (not shown) may use clinician programmer 20 to program therapy for patient 12A, e.g., specify a number of therapy parameter sets and provide the parameter sets to IMD 14A. The clinician may also use clinician programmer 20 to retrieve information collected by IMD 14A. The clinician may use clinician programmer 20 to communicate with IMD 14A both during initial programming of IMD 14A, and for collection of information and further programming during follow-up visits.

Clinician programmer 20 may, as shown in FIG. 1A, be a handheld computing device. Clinician programmer 20 includes a display 22, such as a LCD or LED display, to display information to a user. Clinician programmer 20 may also include a keypad 24, which may be used by a user to interact with clinician programmer 20. In some embodiments, display 22 may be a touch screen display, and a user may interact with clinician programmer 20 via display 22. A user may also interact with clinician programmer 20 using peripheral pointing devices, such as a stylus or mouse. Keypad 24 may take the form of an alphanumeric keypad or a reduced set of keys associated with particular functions.

Systems 10 may also includes a patient programmer 26 (illustrated as part of system 10A in FIG. 1A), which also may, as shown in FIG. 1A, be a handheld computing device. Patient 12A may use patient programmer 26 to control the delivery of therapy by IMD 14A. For example, using patient programmer 26, patient 12A may select a current therapy parameter set from among the therapy parameter sets preprogrammed by the clinician, or may adjust one or more parameters of a preprogrammed therapy parameter set to arrive at the current therapy parameter set.

Patient programmer 26 may include a display 28 and a keypad 30, to allow patient 12A to interact with patient programmer 26. In some embodiments, display 28 may be a touch screen display, and patient 12A may interact with patient programmer 26 via display 28. Patient 12A may also interact with patient programmer 26 using peripheral pointing devices, such as a stylus, mouse, or the like.

However, clinician and patient programmers 20, 26 are not limited to the hand-held computer embodiments illustrated in FIG. 1A. Programmers 20, 26 according to the invention may be any sort of computing device. For example, a programmer 20, 26 according to the invention may be a tablet-based computing device, a desktop computing device, or a workstation.

IMDs 14, clinician programmers 20 and patient programmers 26 may, as shown in FIG. 1A, communicate via wireless communication. Clinician programmer 20 and patient programmer 26 may, for example, communicate via wireless communication with IMD 14A using radio frequency (RF) telemetry techniques known in the art. Clinician programmer 20 and patient programmer 26 may communicate with each other using any of a variety of local wireless communication techniques, such as RF communication according to the 802.11 or Bluetooth specification sets, infrared communication according to the IRDA specification set, or other standard or proprietary telemetry protocols.

Clinician programmer 20 and patient programmer 26 need not communicate wirelessly, however. For example, programmers 20 and 26 may communicate via a wired connection, such as via a serial communication cable, or via exchange of removable media, such as magnetic or optical disks, or memory cards or sticks. Further, clinician programmer 20 may communicate with one or both of IMD 14 and patient programmer 26 via remote telemetry techniques known in the art, communicating via a local area network (LAN), wide area network (WAN), public switched telephone network (PSTN), or cellular telephone network, for example.

As mentioned above, IMDs 14 collect information relating to the quality of sleep experienced by patients 12. Specifically, as will be described in greater detail below, IMDs 14 monitor one or more physiological parameters of patients 12, and determine values for one or more metrics that indicate the quality of sleep based on values of the physiological parameters. Example physiological parameters that IMDs 14 may monitor include activity level, posture, heart rate, ECG morphology, respiration rate, respiratory volume, blood pressure, blood oxygen saturation, partial pressure of oxygen within blood, partial pressure of oxygen within cerebrospinal fluid (CSF), muscular activity and tone, core temperature, subcutaneous temperature, arterial blood flow, the level of melatonin within one or more bodily fluids, brain electrical activity, and eye motion. In some external medical device embodiments of the invention, galvanic skin response may additionally or alternatively be monitored. Further, in some embodiments, IMDs 14 additionally or alternatively monitor the variability of one or more of these parameters. In order to monitor one or more of these parameters, IMDs 14 may include or be coupled to one or more sensors (not shown in FIG. 1), each of which generates a signal as a function of one or more of these physiological parameters.

For example, IMDs 14 may determine sleep efficiency and/or sleep latency values. Sleep efficiency and sleep latency are example sleep quality metrics. IMDs 14 may measure sleep efficiency as the percentage of time while a patient 12 is attempting to sleep that the patient 12 is actually asleep. IMDs 14 may measure sleep latency as the amount of time between a first time when a patient 12 begins attempting to fall asleep and a second time when the patient 12 falls asleep.

IMDs 14 may identify the time at which patient begins attempting to fall asleep in a variety of ways. For example, IMDs 14 may receive an indication from the patient that the patient is trying to fall asleep via patient programmer 26. In other embodiments, IMDs 14 may monitor the activity level of a patient 12, and identify the time when the patient 12 is attempting to fall asleep by determining whether the patient 12 has remained inactive for a threshold period of time, and identifying the time at which the patient 12 became inactive. In still other embodiments, IMDs 14 may monitor the posture of a patient 12, and may identify the time when the patient 12 becomes recumbent, e.g., lies down, as the time when the patient 12 is attempting to fall asleep. In these embodiments, IMD 14 may also monitor the activity level of the patient 12, and confirm that the patient 12 is attempting to sleep based on the activity level.

As another example, IMDs 14 may determine the time at which a patient 12 is attempting to fall asleep based on the level of melatonin within one or more bodily fluids of the patient 12, such as the patient's blood, cerebrospinal fluid (CSF), or interstitial fluid. IMDs 14 may also determine a melatonin level based on metabolites of melatonin located in the saliva or urine of the patient. Melatonin is a hormone secreted by the pineal gland into the bloodstream and the CSF as a function of exposure of the optic nerve to light, which synchronizes the patient's circadian rhythm. In particular, increased levels of melatonin during evening hours may cause physiological changes in a patient 12, which, in turn, may cause the patient 12 to attempt to fall asleep.

IMDs 14 may, for example, detect an increase in the level of melatonin in a bodily fluid, and estimate the time that a patient 12 will attempt to fall asleep based on the detection. For example, IMDs 14 may compare the melatonin level or rate of change in the melatonin level to a threshold level, and identify the time that threshold value is exceeded. IMDs 14 may identify the time that a patient 12 is attempting to fall asleep as the time that the threshold is exceeded, or some amount of time after the threshold is exceeded.

IMDs 14 may identify the time at which a patient 12 has fallen asleep based on the activity level of the patient and/or one or more of the other physiological parameters that may be monitored by IMDs 14 as indicated above. For example, IMDs 14 may identify a discernable change, e.g., a decrease, in one or more physiological parameters, or the variability of one or more physiological parameters, which may indicate that a patient 12 has fallen asleep. In some embodiments, IMDs 14 determine a sleep probability metric value based on a value of a physiological parameter monitored by the medical device. In such embodiments, the sleep probability metric value may be compared to a threshold to identify when the patient has fallen asleep. In some embodiments, a sleep probability metric value is determined based on a value of each of a plurality of physiological parameters, the sleep probability values are averaged or otherwise combined to provide an overall sleep probability metric value, and the overall sleep probability metric value is compared to a threshold to identify the time that the patient falls asleep.

Other sleep quality metrics include total time sleeping per day, and the amount or percentage of time sleeping during nighttime or daytime hours per day. In some embodiments, IMDs 14 may be able to detect arousal events and apneas occurring during sleep based on one or more monitored physiological parameters, and the number of apnea and/or arousal events per night may be determined as a sleep quality metric. Further, in some embodiments IMDs 14 may be able to determine which sleep state patient 12 is in based on one or more monitored physiological parameters, e.g., rapid eye movement (REM), S1, S2, S3, or S4, and the amount of time per day spent in these various sleep states may be a sleep quality metric.

The S3 and S4 sleep states may be of particular importance to the quality of sleep experienced by patients 12. Interruption from reaching these states, or inadequate time per night spent in these states, may cause patients 12 to not feel rested. For this reason, the S3 and S4 sleep states are believed to provide the “refreshing” part of sleep.

In some cases, interruption from reaching the S3 and S4 sleep states, or inadequate time per night spent in these states has been demonstrated to cause normal subjects to exhibit some symptoms of fibromyalgia. Also, subjects with fibromyalgia usually do not reach these sleep states. For these reasons, in some embodiments, IMDs 14 may determine an amount or percentage of time spent in one or both of the S3 and S4 sleep states as a sleep quality metric.

In some embodiments, IMDs 14 may determine average or median values of one or more sleep quality metrics over greater periods of time, e.g., a week or a month, as the value of the sleep quality metric. Further, in embodiments in which IMDs 14 collect values for a plurality of the sleep quality metrics identified above, IMDs 14 may determine a value for an overall sleep quality metric based on the collected values for the plurality of sleep quality metrics. IMDs 14 may determine the value of an overall sleep quality metric by applying a function or look-up table to a plurality of sleep quality metric values, which may also include the application of weighting factors to one or more of the individual sleep quality metric values.

In some embodiments, IMDs 14 may identify the current set of therapy parameters when a value of one or more sleep quality metrics is collected, and may associate that value with the current therapy parameter sets. For example, for each of a plurality therapy parameter sets used over time by an IMD 14 to deliver therapy to a patient 12, the IMD 14 may store a representative value of each of one or more sleep quality metrics in a memory with an indication of the therapy parameter set with which that representative value is associated. A representative value of sleep quality metric for a therapy parameter set may be the mean or median of collected sleep quality metric values that have been associated with that therapy parameter set.

One or both of programmers 20, 26 may receive sleep quality metric values from an IMD 14, and may provide sleep quality information to a user based on the sleep quality metric values. For example, a patient programmer 26 may provide a message to a patient 12, e.g., via display 28, related to sleep quality based on received sleep quality metric values. A patient programmer 26 may, for example, suggest that a patient 12 visit a clinician for prescription of sleep medication or for an adjustment to the therapy delivered by an IMD 14. As other examples, a patient programmer 26 may suggest that a patient 12 increase the intensity of therapy delivered by an IMD 14 during nighttime hours relative to previous nights, or select a different therapy parameter set for use by the IMD 14 than the patient had selected during previous nights. Further, a patient programmer 26 may report the quality of the patient's sleep to a patient 12 to, for example, provide a patient 12 with an objective indication of whether his or her sleep quality is good, adequate, or poor.

A clinician programmer 20 may receive sleep quality metric values from an IMD 14, and present a variety of types of sleep information to a clinician, e.g., via display 22, based on the sleep quality metric values. For example, a clinician programmer 20 may present a graphical representation of the sleep quality metric values, such as a trend diagram of values of one or more sleep quality metrics over time, or a histogram or pie chart illustrating percentages of time that a sleep quality metric was within various value ranges.

In embodiments in which an IMD 14 associates sleep quality metric values with therapy parameter sets, a clinician programmer 20 may receive representative values for one or more sleep quality metrics from the IMD 14 and information identifying the therapy parameter sets with which the representative values are associated. Using this information, the clinician programmer 20 may display a list of the therapy parameter sets to the clinician ordered according to their associated representative sleep quality metric values. The clinician may use such a list to identify effective or ineffective therapy parameter sets. Where a plurality of sleep quality metric values are collected, a clinician programmer 20 may order the list according to values of a user-selected one of the sleep quality metrics. In this manner, the clinician may quickly identify the therapy parameter sets producing the best results in terms of sleep quality.

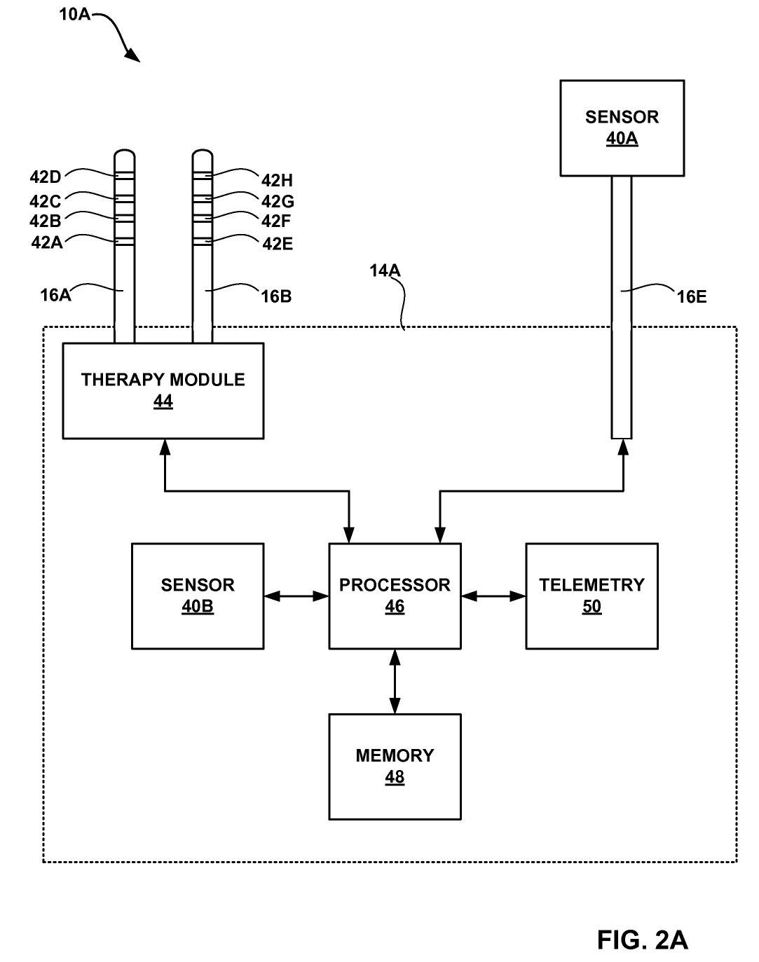

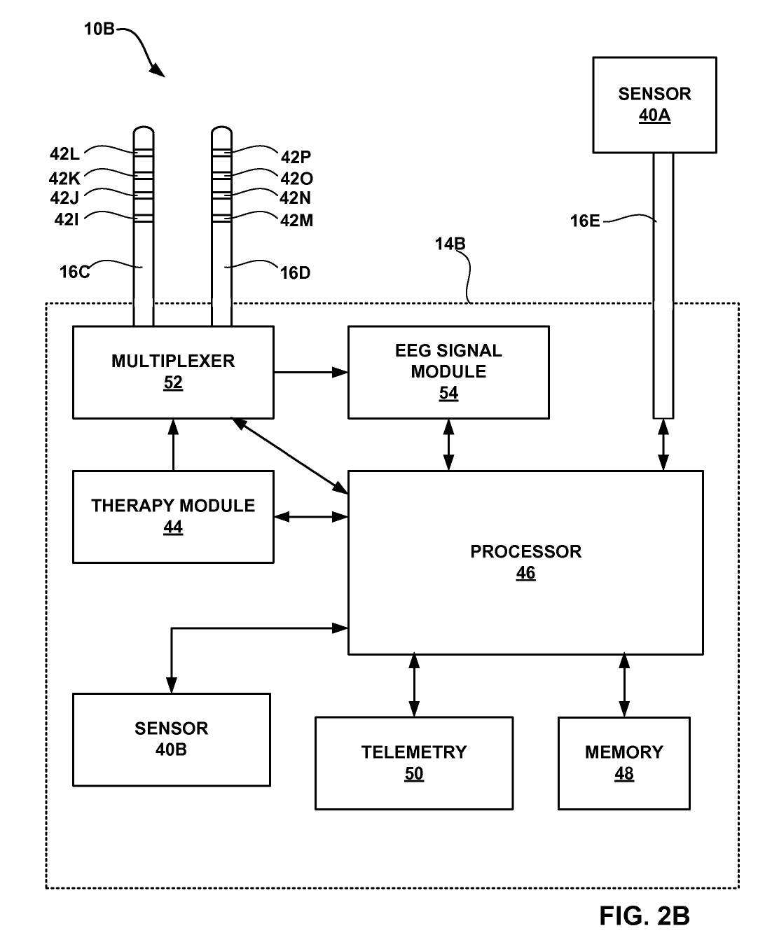

FIGS. 2A and 2B are block diagrams further illustrating systems 10A and 10B. In particular, FIG. 2A illustrates an example configuration of IMD 14A and leads 16A and 16B. FIG. 2B illustrates an example configuration of IMD 14B and leads 16C and 16D. FIGS. 2A and 2B also illustrate sensors 40A and 40B (collectively “sensors 40”) that generate signals as a function of one or more physiological parameters of patients 12. As will be described in greater detail below, IMDs 14 monitor the signals to determine values for one or more metrics that are indicative of sleep quality.

IMD 14A may deliver neurostimulation therapy via electrodes 42A-D of lead 16A and electrodes 42E-H of lead 16B, while IMD 14B delivers neurostimulation via electrodes 421-L of lead 16C and electrodes 42 M-P of lead 16D (collectively “electrodes 42”). Electrodes 42 may be ring electrodes. The configuration, type and number of electrodes 42 illustrated in FIGS. 2A and 2B are merely exemplary. For example, leads 16 may each include eight electrodes 42, and the electrodes 42 need not be arranged linearly on each of leads 16.

In each of systems 10A and 10B, electrodes 42 are electrically coupled to a therapy delivery module 44 via leads 16. Therapy delivery module 44 may, for example, include an output pulse generator coupled to a power source such as a battery. Therapy delivery module 44 may deliver electrical pulses to a patient 12 via at least some of electrodes 42 under the control of a processor 46, which controls therapy delivery module 44 to deliver neurostimulation therapy according to a current therapy parameter set. However, the invention is not limited to implantable neurostimulator embodiments or even to IMDs that deliver electrical stimulation. For example, in some embodiments a therapy delivery module 44 of an IMD may include a pump, circuitry to control the pump, and a reservoir to store a therapeutic agent for delivery via the pump.

Processor 46 may include a microprocessor, a controller, a digital signal processor (DSP), an application specific integrated circuit (ASIC), a field-programmable gate array (FPGA), discrete logic circuitry, or the like. Memory 48 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, and the like. In some embodiments, memory 48 stores program instructions that, when executed by processor 46, cause IMD 14 and processor 46 to perform the functions attributed to them herein.

Each of sensors 40 generates a signal as a function of one or more physiological parameters of a patient 12. IMDs 14 may include circuitry (not shown) that conditions the signals generated by sensors 40 such that they may be analyzed by processor 46. For example, IMDs 14 may include one or more analog to digital converters to convert analog signals generated by sensors 40 into digital signals usable by processor 46, as well as suitable filter and amplifier circuitry. Although shown as including two sensors 40, systems 10A and 10B may include any number of sensors.

Further, as illustrated in FIGS. 2A and 2B, sensors 40 may be included as part of IMDs 14, or coupled to IMDs 14 via leads 16. Sensors 40 may be coupled to IMD 14 via therapy leads 16A-16D, or via other leads 16, such as lead 16E depicted in FIGS. 2A and 2B. In some embodiments, a sensor 40 located outside of an IMD 14 may be in wireless communication with processor 46. Wireless communication between sensors 40 and IMDs 14 may, as examples, include RF communication or communication via electrical signals conducted through the tissue and/or fluid of a patient 12.

As discussed above, exemplary physiological parameters of patients 12 that may be monitored by IMDs 14 to determine values of one or more sleep quality metrics include activity level, posture, heart rate, ECG morphology, respiration rate, respiratory volume, blood pressure, blood oxygen saturation, partial pressure of oxygen within blood, partial pressure of oxygen within cerebrospinal fluid, muscular activity and tone, core temperature, subcutaneous temperature, arterial blood flow, the level of melatonin within a bodily fluid of patients 12, electrical activity of the brain of the patient, and eye motion. Further, as discussed above, in some external medical device embodiments of the invention, galvanic skin response may additionally or alternatively be monitored. Sensors 40 may be of any type known in the art capable of generating a signal as a function of one or more of these parameters.

In some embodiments, in order to determine one or more sleep quality metric values, processor 46 determines when a patient 12 is attempting to fall asleep. For example, processor 46 may identify the time that the patient begins attempting to fall asleep based on an indication received from the patient, e.g., via patient programmer 26 and a telemetry circuit 50. In other embodiments, processor 46 identifies the time that a patient 12 begins attempting to fall asleep based on the activity level of the patient 12.

In such embodiments, an IMD 14 may include one or more sensors 40 that generate a signal as a function of patient activity. For example, sensors 40 may include one or more accelerometers, gyros, mercury switches, or bonded piezoelectric crystals that generates a signal as a function of patient activity, e.g., body motion, footfalls or other impact events, and the like. Additionally or alternatively, sensors 40 may include one or more electrodes that generate an electromyogram (EMG) signal as a function of muscle electrical activity, which may indicate the activity level of a patient. The electrodes may be, for example, located in the legs, abdomen, chest, back or buttocks of a patient 12 to detect muscle activity associated with walking, running, or the like. The electrodes may be coupled to the IMD 14 wirelessly or by leads 16 or, if IMD 14 is implanted in these locations, integrated with a housing of the IMD 14.

However, bonded piezoelectric crystals located in these areas generate signals as a function of muscle contraction in addition to body motion, footfalls or other impact events. Consequently, use of bonded piezoelectric crystals to detect activity of a patient 12 may be preferred in some embodiments in which it is desired to detect muscle activity in addition to body motion, footfalls, or other impact events. Bonded piezoelectric crystals may be coupled to an IMD 14 wirelessly or via leads 16, or piezoelectric crystals may be bonded to the can of the IMD 14 when the IMD is implanted in these areas, e.g., in the back, chest, buttocks or abdomen of the patient 12.

Processor 46 may identify a time when the activity level of a patient 12 falls below a threshold activity level value stored in memory 48, and may determine whether the activity level remains substantially below the threshold activity level value for a threshold amount of time stored in memory 48. In other words, a patient 12 remaining inactive for a sufficient period of time may indicate that the patient 12 is attempting to fall asleep. If processor 46 determines that the threshold amount of time is exceeded, processor 46 may identify the time at which the activity level fell below the threshold activity level value as the time that the patient 12 began attempting to fall asleep.

In some embodiments, processor 46 determines whether a patient 12 is attempting to fall asleep based on whether the patient 12 is or is not recumbent, e.g., lying down. In such embodiments, sensors 40 may include a plurality of accelerometers, gyros, or magnetometers oriented orthogonally that generate signals which indicate the posture of the patient 12. In addition to being oriented orthogonally with respect to each other, each of sensors 40 used to detect the posture of the patient 12 may be generally aligned with an axis of the body of the patient 12. In exemplary embodiments, an IMD 14 includes three orthogonally oriented posture sensors 40.

When sensors 40 include accelerometers, for example, that are aligned in this manner, processor 46 may monitor the magnitude and polarity of DC components of the signals generated by the accelerometers to determine the orientation of the patient 12 relative to the Earth's gravity, e.g., the posture of the patient 12. In particular, the processor 46 may compare the DC components of the signals to respective threshold values stored in memory 48 to determine whether the patient 12 is or is not recumbent. Further information regarding use of orthogonally aligned accelerometers to determine patient posture may be found in a commonly assigned U.S. Pat. No. 5,593,431, which issued to Todd J. Sheldon. Other sensors 40 that may generate a signal that indicates the posture of a patient 12 include electrodes that generate an electromyogram (EMG) signal, or bonded piezoelectric crystals that generate a signal as a function of contraction of muscles. Such sensors 40 may be implanted in the legs, buttocks, chest, abdomen, or back of a patient 12, as described above. The signals generated by such sensors when implanted in these locations may vary based on the posture of the patient 12, e.g., may vary based on whether the patient is standing, sitting, or laying down.

Further, the posture of a patient 12 may affect the thoracic impedance of the patient. Consequently, sensors 40 may include an electrode pair, including one electrode integrated with the housing of an IMD 14 and one of electrodes 42, that generates a signal as a function of the thoracic impedance of the patient 12, and processor 46 may detect the posture or posture changes of patient 12 based on the signal. The electrodes of the pair may be located on opposite sides of the patient's thorax. For example, the electrode pair may include one of electrodes 42 located proximate to the spine of a patient for delivery of SCS therapy, and the IMD 14 with an electrode integrated in its housing may be implanted in the abdomen of the patient 12.

Additionally, changes of the posture of a patient 12 may cause pressure changes with the cerebrospinal fluid (CSF) of the patient. Consequently, sensors 40 may include pressure sensors coupled to one or more intrathecal or intracerebroventricular catheters, or pressure sensors coupled to an IMD 14 wirelessly or via lead 16. CSF pressure changes associated with posture changes may be particularly evident within the brain of the patient, e.g., may be particularly apparent in an intracranial pressure (ICP) waveform.

In some embodiments, processor 46 considers both the posture and the activity level of a patient 12 when determining whether patient 12 is attempting to fall asleep. For example, processor 46 may determine whether a patient 12 is attempting to fall asleep based on a sufficiently long period of sub-threshold activity, as described above, and may identify the time that patient began attempting to fall asleep as the time when the patient 12 became recumbent.

In other embodiments, processor 46 determines when a patient 12 is attempting to fall asleep based on the level of melatonin in a bodily fluid. In such embodiments, a sensor 40 may take the form of a chemical sensor that is sensitive to the level of melatonin or a metabolite of melatonin in the bodily fluid, and estimate the time that a patient 12 will attempt to fall asleep based on the detection. For example, processor 46 may compare the melatonin level or rate of change in the melatonin level to a threshold level stored in memory 48, and identify the time that threshold value is exceeded. Processor 46 may identify the time that the patient 12 is attempting to fall asleep as the time that the threshold is exceeded, or some amount of time after the threshold is exceeded. Any of a variety of combinations or variations of the above-described techniques may be used to determine when the patient 12 is attempting to fall asleep, and a specific one or more techniques may be selected based on the sleeping and activity habits of a particular patient.

Processor 46 may also determine when a patient 12 is asleep, e.g., identify the times that the patient 12 falls asleep and wakes up, in order to determine one or more sleep quality metric values. The detected values of physiological parameters of a patient 12, such as activity level, heart rate, ECG morphological features, respiration rate, respiratory volume, blood pressure, blood oxygen saturation, partial pressure of oxygen within blood, partial pressure of oxygen within cerebrospinal fluid, muscular activity and tone, core temperature, subcutaneous temperature, arterial blood flow, brain electrical activity, eye motion and galvanic skin response may discernibly change when the patient 12 falls asleep or wakes up. Some of these physiological parameters may be at low values when patient 12 is asleep. Further, the variability of at least some of these parameters, such as heart rate and respiration rate, may be at a low value when the patient is asleep.

Consequently, in order to detect when a patient 12 falls asleep and wakes up, processor 46 may monitor one or more of these physiological parameters, or the variability of these physiological parameters, and detect the discernable changes in their values associated with a transition between a sleeping state and an awake state. In some embodiments, processor 46 may determine a mean or median value for a parameter based on values of a signal over time, and determine whether a patient 12 is asleep or awake based on the mean or median value. Processor 46 may compare one or more parameter or parameter variability values to thresholds stored in memory 48 to detect when a patient 12 falls asleep or awakes. The thresholds may be absolute values of a physiological parameter, or time rate of change values for the physiological parameter, e.g., to detect sudden changes in the value of a parameter or parameter variability. In some embodiments, a threshold used by processor 46 to determine whether a patient 12 is asleep may include a time component. For example, a threshold may require that a physiological parameter be above or below a threshold value for a period of time before processor 46 determines that patient is awake or asleep.

In some embodiments, in order to determine whether a patient 12 is asleep, processor 46 monitors a plurality of physiological parameters, and determines a value of a metric that indicates the probability that the patient 12 is asleep for each of the parameters based on a value of the parameter. In particular, the processor 46 may apply a function or look-up table to the current, mean or median value, and/or the variability of each of a plurality of physiological parameters to determine a sleep probability metric for each of the plurality of physiological parameters. A sleep probability metric value may be a numeric value, and in some embodiments may be a probability value, e.g., a number within the range from 0 to 1, or a percentage value.

Processor 46 may average or otherwise combine the plurality of sleep probability metric values to provide an overall sleep probability metric value. In some embodiments, processor 46 may apply a weighting factor to one or more of the sleep probability metric values prior to combination. Processor 46 may compare the overall sleep probability metric value to one or more threshold values stored in memory 48 to determine when the patient 12 falls asleep or awakes. Use of sleep probability metric values to determine when a patient is asleep based on a plurality of monitored physiological parameters is described in greater detail in a commonly-assigned and copending U.S. patent application Ser. No. 11/081,786 by Ken Heruth and Keith Miesel, entitled “DETECTING SLEEP,” which was assigned Attorney Docket No. 1023-360US02 and filed on Mar. 16, 2005, and is incorporated herein by reference in its entirety.

To enable processor 46 to determine when a patient 12 is asleep or awake, sensors 40 may include, for example, activity sensors as described above. In some embodiments, the activity sensors may include electrodes or bonded piezoelectric crystals, which may be implanted in the back, chest, buttocks, or abdomen of a patient 12 as described above. In such embodiments, processor 46 may detect the electrical activation and contractions of muscles associated with gross motor activity of the patient, e.g., walking, running or the like via the signals generated by such sensors. Processor 46 may also detect spasmodic, irregular, movement disorder or pain related muscle activation via the signals generated by such sensors. Such muscle activation may indicate that a patient 12 is not sleeping, e.g., unable to sleep, or if a patient 12 is sleeping, may indicate a lower level of sleep quality.

As another example, sensors 40 may include electrodes located on leads or integrated as part of the housing of an IMD 14 that generate an electrogram signal as a function of electrical activity of the heart of a patient 12, and processor 46 may monitor the heart rate of the patient 12 based on the electrogram signal. In other embodiments, a sensor may include an acoustic sensor within an IMD 14, a pressure or flow sensor within the bloodstream or cerebrospinal fluid of a patient 12, or a temperature sensor located within the bloodstream of a patient 12. The signals generated by such sensors may vary as a function of contraction of the heart of a patient 12, and can be used by an IMD 14 to monitor the heart rate of a patient 12.

In some embodiments, processor 46 may detect, and measure values for one or more ECG morphological features within an electrogram generated by electrodes as described above. ECG morphological features may vary in a manner that indicates whether a patient 12 is asleep or awake. For example, the amplitude of the ST segment of the ECG may decrease when a patient 12 is asleep. Further, the amplitude of QRS complex or T-wave may decrease, and the widths of the QRS complex and T-wave may increase when a patient 12 is asleep. The QT interval and the latency of an evoked response may increase when a patient 12 is asleep, and the amplitude of the evoked response may decrease when a patient 12 is asleep.

In some embodiments, sensors 40 may include an electrode pair, including one electrode integrated with the housing of an IMD 14 and one of electrodes 42, that generates a signal as a function of the thoracic impedance of a patient 12, as described above. The thoracic impedance signal varies as a function of respiration by patient 12. In other embodiments, sensors 40 may include a strain gage, bonded piezoelectric element, or pressure sensor within the blood or cerebrospinal fluid that generates a signal that varies based on patient respiration. An electrogram generated by electrodes as discussed above may also be modulated by patient respiration, and may be used as an indirect representation of respiration rate.

Sensors 40 may include electrodes that generate an electromyogram (EMG) signal as a function of muscle electrical activity, as described above, or may include any of a variety of known temperature sensors to generate a signal as a function of a core or subcutaneous temperature of a patient 12. Such electrodes and temperature sensors may be incorporated within the housing of an IMD 14, or coupled to the IMD 14 wirelessly via leads. Sensors 40 may also include a pressure sensor within, or in contact with, a blood vessel. The pressure sensor may generate a signal as a function of the a blood pressure of a patient 12, and may, for example, comprise a Chronicle Hemodynamic Monitor™ commercially available from Medtronic, Inc. of Minneapolis, Minn. Further, certain muscles of a patient 12, such as the muscles of the patient's neck, may discernibly relax when the patient 12 is asleep or within certain sleep states. Consequently, sensors 40 may include strain gauges or EMG electrodes implanted in such locations that generate a signal as a function of muscle tone.

Sensors 40 may also include optical pulse oximetry sensors or Clark dissolved oxygen sensors located within, as part of a housing of, or outside of an IMD 14, which generate signals as a function of blood oxygen saturation and blood oxygen partial pressure respectively. In some embodiments, a system 10 may include a catheter with a distal portion located within the cerebrospinal fluid of a patient 12, and the distal end may include a Clark dissolved oxygen sensor to generate a signal as a function of the partial pressure of oxygen within the cerebrospinal fluid. Embodiments in which an IMD comprises an implantable pump, for example, may include a catheter with a distal portion located in the cerebrospinal fluid.

In some embodiments, sensors 40 may include one or more intraluminal, extraluminal, or external flow sensors positioned to generate a signal as a function of arterial blood flow. A flow sensor may be, for example, an electromagnetic, thermal convection, ultrasonic-Doppler, or laser-Doppler flow sensor. Further, in some external medical device embodiments of the invention, sensors 40 may include one or more electrodes positioned on the skin of a patient 12 to generate a signal as a function of galvanic skin response.

Additionally, in some embodiments, sensors 40 may include one or more electrodes positioned within or proximate to the brain of patient 12, which detect electrical activity of the brain. For example, in embodiments in which an IMD 14 delivers stimulation or therapeutic agents to the brain, processor 46 may be coupled to electrodes implanted on or within the brain via a lead 16. System 10B, illustrated in FIGS. 1B and 2B, is an example of a system that includes electrodes 42, located on or within the brain of patient 12B, that are coupled to IMD 14B.

As shown in FIG. 2B, electrodes 42 may be selectively coupled to therapy module 44 or an EEG signal module 54 by a multiplexer 52, which operates under the control of processor 46. EEG signal module 54 receives signals from a selected set of the electrodes 42 via multiplexer 52 as controlled by processor 46. EEG signal module 54 may analyze the EEG signal for certain features indicative of sleep or different sleep states, and provide indications of relating to sleep or sleep states to processor 46. Thus, electrodes 42 and EEG signal module 54 may be considered another sensor 40 in system 10B. IMD 14B may include circuitry (not shown) that conditions the EEG signal such that it may be analyzed by processor 52. For example, IMD 14B may include one or more analog to digital converters to convert analog signals received from electrodes 42 into digital signals usable by processor 46, as well as suitable filter and amplifier circuitry.

In some embodiments, processor 46 will only request EEG signal module 54 to operate when one or more other physiological parameters indicate that patient 12B is already asleep. However, processor 46 may also direct EEG signal module to analyze the EEG signal to determine whether patient 12B is sleeping, and such analysis may be considered alone or in combination with other physiological parameters to determine whether patient 12B is asleep. EEG signal module 60 may process the EEG signals to detect when patient 12 is asleep using any of a variety of techniques, such as techniques that identify whether a patient is asleep based on the amplitude and/or frequency of the EEG signals. In some embodiments, the functionality of EEG signal module 54 may be provided by processor 46, which, as described above, may include one or more microprocessors, ASICs, or the like.

In other embodiments, processor 46 may be wirelessly coupled to electrodes that detect brain electrical activity. For example, one or more modules may be implanted beneath the scalp of the patient, each module including a housing, one or more electrodes, and circuitry to wirelessly transmit the signals detected by the one or more electrodes to an IMD 14. In other embodiments, the electrodes may be applied to the patient's scalp, and electrically coupled to a module that includes circuitry for wirelessly transmitting the signals detected by the electrodes to an IMD 14. The electrodes may be glued to the patient's scalp, or a head band, hair net, cap, or the like may incorporate the electrodes and the module, and may be worn by a patient 12 to apply the electrodes to the patient's scalp when, for example, the patient is attempting to sleep. The signals detected by the electrodes and transmitted to an IMD 14 may be EEG signals, and processor 46 may process the EEG signals to detect when the patient 12 is asleep using any of a variety of known techniques, such as techniques that identify whether a patient is asleep based on the amplitude and/or frequency of the EEG signals.

Also, the motion of the eyes of a patient 12 may vary depending on whether the patient is sleeping and which sleep state the patient is in. Consequently, sensors 40 may include electrodes place proximate to the eyes of a patient 12 to detect electrical activity associated with motion of the eyes, e.g., to generate an electro-oculography (EOG) signal. Such electrodes may be coupled to an IMD 14 via one or more leads 16, or may be included within modules that include circuitry to wirelessly transmit detected signals to the IMD 14. Wirelessly coupled modules incorporating electrodes to detect eye motion may be worn externally by a patient 12, e.g., attached to the skin of the patient 12 proximate to the eyes by an adhesive when the patient is attempting to sleep.

Processor 46 may also detect arousals and/or apneas that occur when a patient 12 is asleep based on one or more of the above-identified physiological parameters. For example, processor 46 may detect an arousal based on an increase or sudden increase in one or more of heart rate, heart rate variability, respiration rate, respiration rate variability, blood pressure, or muscular activity as the occurrence of an arousal. Processor 46 may detect an apnea based on a disturbance in the respiration rate of a patient 12, e.g., a period with no respiration.

Processor 46 may also detect arousals or apneas based on sudden changes in one or more of the ECG morphological features identified above. For example, a sudden elevation of the ST segment within the ECG may indicate an arousal or an apnea. Further, sudden changes in the amplitude or frequency of an EEG signal, EOG signal, or muscle tone signal may indicate an apnea or arousal. Memory 48 may store thresholds used by processor 46 to detect arousals and apneas. Processor 46 may determine, as a sleep quality metric value, the number of apnea events and/or arousals during a night.

Further, in some embodiments, processor 46 may determine which sleep state patient 12 is in during sleep, e.g., REM, S1, S2, S3, or S4, based on one or more of the monitored physiological parameters. In some embodiments, memory 48 may store one or more thresholds for each of sleep states, and processor 46 may compare physiological parameter or sleep probability metric values to the thresholds to determine which sleep state patient 12 is currently in. Further, in some embodiments, processor 46 and/or EEG signal module 54 may use any of a variety of known techniques for determining which sleep state patient is in based on an EEG signal, which processor 46 and/or EEG signal module may receive via electrodes 42 as described above, such as techniques that identify sleep state based on the amplitude and/or frequency of the EEG signals. In some embodiments, processor 46 may also determine which sleep state patient is in based on an EOG signal, which processor 46 may receive via electrodes as described above, either alone or in combination with an EEG signal, using any of a variety of techniques known in the art. Processor 46 may determine, as sleep quality metric values, the amounts of time per night spent in the various sleep states. As discussed above, inadequate time spent in deeper sleep states, e.g., S3 and S4, is an indicator of poor sleep quality. Consequently, in some embodiments, processor 46 may determine an amount or percentage of time spent in one or both of the S3 and S4 sleep states as a sleep quality metric.

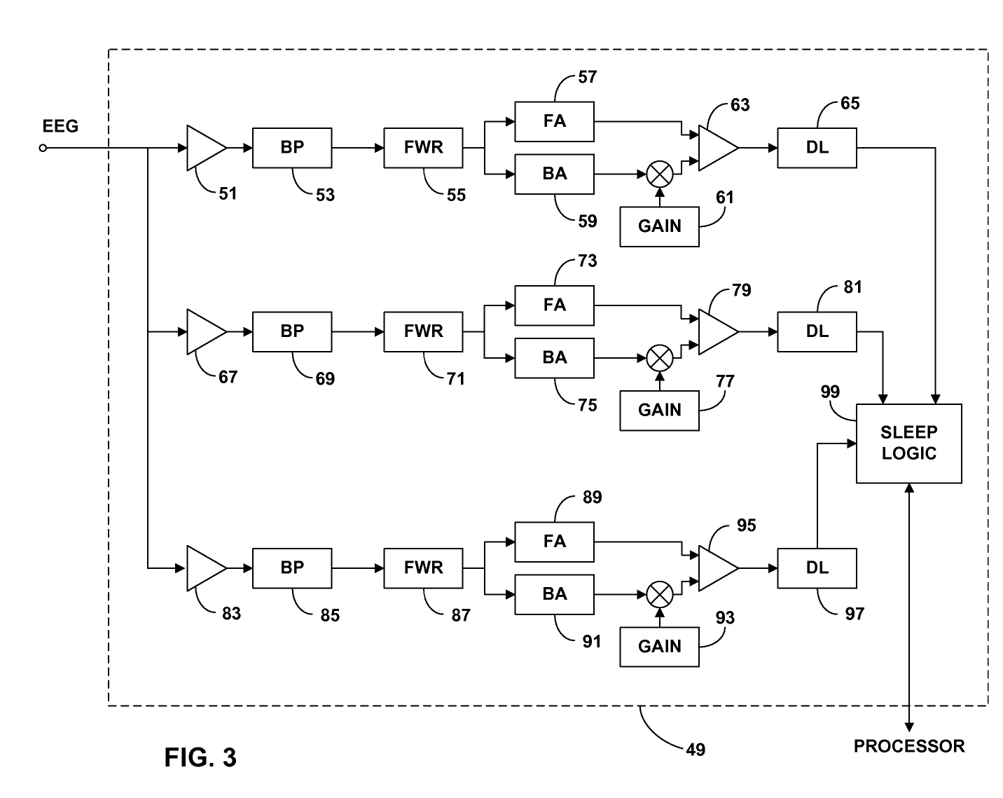

FIG. 3 is a logical diagram of an example circuit that detects the sleep type of a patient based on the electroencephalogram (EEG) signal. Module 49, shown in FIG. 3, may be integrated into an EEG signal module 54 of IMD 14B, or some other implantable or external device capable of detecting an EEG signal according to other embodiments of the invention. In such embodiments, module 49 may be used to, for example, determine whether a patient 12 is asleep, or in which sleep state the patient is.

An EEG signal detected by electrodes 42 adjacent to the brain 19 of patent 12B is transmitted into module 49 and provided to three channels, each of which includes a respective one of amplifiers 51, 67 and 83, and bandpass filters 53, 69 and 85. In other embodiments, a common amplifier amplifies the EEG signal prior to filters 53, 69 and 85. Bandpass filter 53 allows frequencies between approximately 4 Hz and approximately 8 Hz, and signals within the frequency range may be prevalent in the EEG during S1 and S2 sleep states. Bandpass filter 69 allows frequencies between approximately 1 Hz and approximately 3 Hz, which may be prevalent in the EEG during the S3 and S4 sleep states. Bandpass filter 85 allows frequencies between approximately 10 Hz and approximately 50 Hz, which may be prevalent in the EEG during REM sleep. Each resulting signal may then be processed to identify in which sleep state patient 12B is.

After bandpass filtering of the original EEG signal, the filtered signals are similarly processed in parallel before being delivered to sleep logic module 99. For ease of discussion, only one of the three channels will be discussed herein, but each of the filtered signals may be processed similarly.

Once the EEG signal is filtered by bandpass filter 53, the signal is rectified by full-wave rectifier 55. Modules 57 and 59 respectively determine the foreground average and background average so that the current energy level can be compared to a background level at comparator 63. The signal from background average is increased by gain 61 before being sent to comparator 63, because comparator 63 operates in the range of millivolts or volts while the EEG signal amplitude is originally on the order of microvolts. The signal from comparator 63 is indicative of sleep stages S1 and S2. If duration logic 65 determines that the signal is greater than a predetermined level for a predetermined amount of time, the signal is sent to sleep logic module 99 indicating that patient 12 may be within the S1 or S2 sleep states. In some embodiments, as least duration logic 65, 81, 97 and sleep logic 99 may be embodied in a processor of the device containing EEG module 49.

Module 49 may detect all sleep types for patient 12. Further, the beginning of sleep may be detected by module 49 based on the sleep state of patient 12. Some of the components of module 49 may vary from the example of FIG. 3. For example, gains 61, 77 and 93 may be provided from the same power source. Module 49 may be embodied as analog circuitry, digital circuitry, or a combination thereof.

In other embodiments, module 49 may not need to reference the background average to determine the current state of sleep of patient 12. Instead, the power of the signals from bandpass filters 53, 69 and 85 are compared to each other, and sleep logic module 99 determines which the sleep state of patient 12 based upon the frequency band that has the highest power. In this case, the signals from full-wave rectifiers 55, 71 and 87 are sent directly to a device that calculates the signal power, such as a spectral power distribution module (SPD), and then to sleep logic module 99 which determines the frequency band of the greatest power, e.g., the sleep state of patient 12B. In some cases, the signal from full-wave rectifiers 55, 71 and 87 may be normalized by a gain component to correctly weight each frequency band.

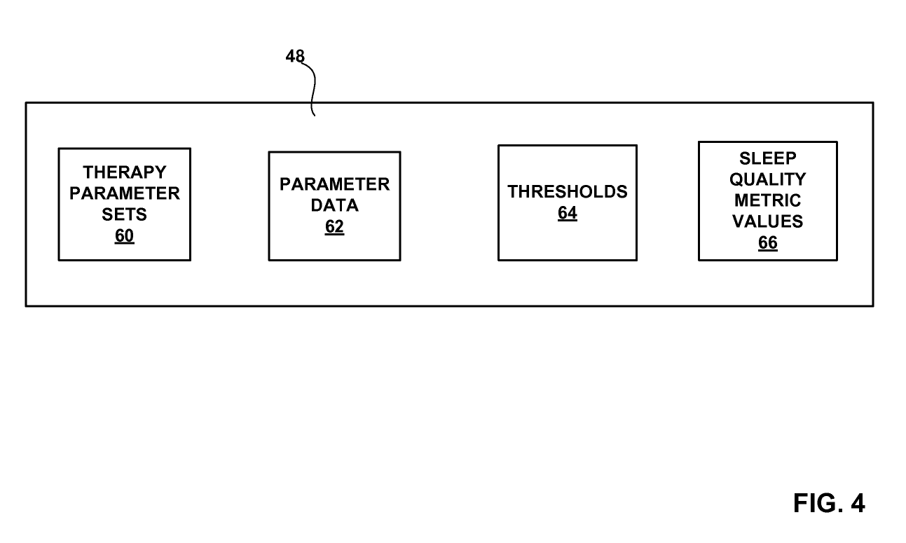

FIG. 4 further illustrates memory 48 of IMDs 14A and 14B. As illustrated in FIG. 4, memory 48 stores information describing a plurality of therapy parameter sets 60. Therapy parameter sets 60 may include parameter sets specified by a clinician using clinician programmer 20. Therapy parameter sets 60 may also include parameter sets that are the result of patient 12 changing one or more parameters of one of the preprogrammed therapy parameter sets via patient programmer 26.

Memory 48 may also include parameter information 62 recorded by processor 46, e.g., physiological parameter values, or mean or median physiological parameter values. Memory 48 stores threshold values 64 used by processor 46 in the collection of sleep quality metric values, as discussed above. In some embodiments, memory 48 also stores one or more functions or look-up tables (not shown) used by processor 46 to determine sleep probability metric values, or to determine an overall sleep quality metric value.

Further, processor 46 stores determined values 66 for one or more sleep quality metrics within memory 48. Processor 46 may collect sleep quality metric values 66 each time patient 12 sleeps, or only during selected times that patient 12 is asleep. Processor 46 may store each sleep quality metric value determined within memory 48 as a sleep quality metric value 66, or may store mean or median sleep quality metric values over periods of time such as weeks or months as sleep quality metric values 66. Further, processor 46 may apply a function or look-up table to a plurality of sleep quality metric values to determine overall sleep quality metric value, and may store the overall sleep quality metric values within memory 48. The application of a function or look-up table by processor 46 for this purpose may involve the use or weighting factors for one or more of the individual sleep quality metric values.

In some embodiments, processor 46 identifies which of therapy parameter sets 60 is currently selected for use in delivering therapy to patient 12 when a value of one or more sleep quality metrics is collected, and may associate that value with the current therapy parameter set. For example, for each of the plurality of therapy parameter sets 60, processor 46 may store a representative value of each of one or more sleep quality metrics within memory 48 as a sleep quality metric value 66 with an indication of which of the therapy parameter sets that representative value is associated with. A representative value of sleep quality metric for a therapy parameter set may be the mean or median of collected sleep quality metric values that have been associated with that therapy parameter set.

Referring again to FIG. 2, IMDs 14 also include a telemetry circuit 50 that allows processor 46 to communicate with a clinician programmer 20 and patient programmer 26. Processor 46 may receive information identifying therapy parameter sets 60 preprogrammed by the clinician and threshold values 64 from clinician programmer 20 via telemetry circuit 50 for storage in memory 48. Processor 46 may receive an indication of the therapy parameter set 60 selected by patient 12 for delivery of therapy, or adjustments to one or more of therapy parameter sets 60 made by patient 12, from patient programmer 26 via telemetry circuit 50. Programmers 20, 26 may receive sleep quality metric values 66 from processor 46 via telemetry circuit 50.

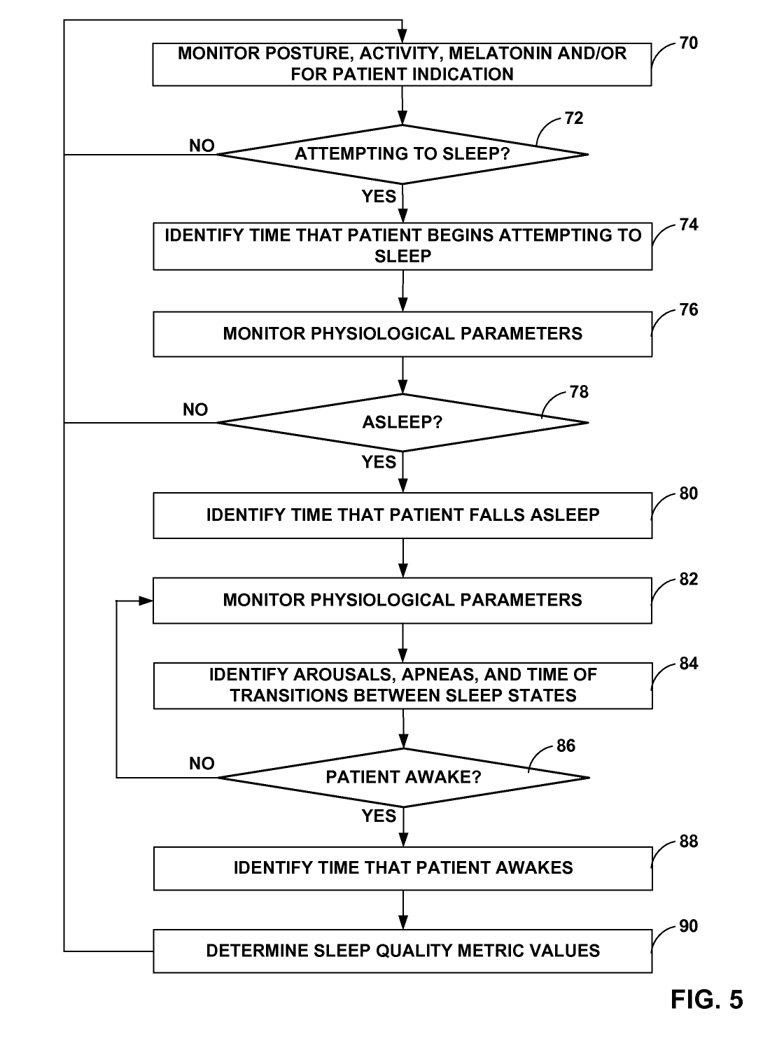

FIG. 5 is a flow diagram illustrating an example method for collecting sleep quality information that may be employed by IMDs 14. An IMD 14 monitors the posture, activity level, and/or melatonin level of a patient 12, or monitors for an indication from patient 12, e.g., via patient programmer 26 (70), and determines whether patient 12 is attempting to fall asleep based on the posture, activity level, melatonin level, and/or a patient indication, as described above (72). If IMD 14 determines that patient 12 is attempting to fall asleep, IMD 14 identifies the time that patient 12 began attempting to fall asleep using any of the techniques described above (74), and monitors one or more of the various physiological parameters of patient 12 discussed above to determine whether patient 12 is asleep (76, 78).

In some embodiments, IMD 14 compares parameter values or parameter variability values to one or more threshold values 64 to determine whether patient 12 is asleep. In other embodiments, IMD 14 applies one or more functions or look-up tables to determine one or more sleep probability metric values based on the physiological parameter values, and compares the sleep probability metric values to one or more threshold values 64 to determine whether patient 12 is asleep. While monitoring physiological parameters (76) to determine whether patient 12 is asleep (78), IMD 14 may continue to monitor the posture and/or activity level of patient 12 (70) to confirm that patient 12 is still attempting to fall asleep (72).

When IMD 14 determines that patient 12 is asleep, e.g., by analysis of the various parameters contemplated herein, IMD 14 will identify the time that patient 12 fell asleep (80). While patient 12 is sleeping, IMD 14 will continue to monitor physiological parameters of patient 12 (82). As discussed above, IMD 14 may identify the occurrence of arousals and/or apneas based on the monitored physiological parameters (84). Further, IMD 14 may identify the time that transitions between sleep states, e.g., REM, S1, S2, S3, and S4, occur based on the monitored physiological parameters (84).

Additionally, while patient 12 is sleeping, IMD 14 monitors physiological parameters of patient 12 (82) to determine whether patient 12 has woken up (86). When IMD 14 determines that patient 12 is awake, IMD 14 identifies the time that patient 12 awoke (88), and determines sleep quality metric values based on the information collected while patient 12 was asleep (90).

For example, one sleep quality metric value IMD 14 may calculate is sleep efficiency, which IMD 14 may calculate as a percentage of time during which patient 12 is attempting to sleep that patient 12 is actually asleep. IMD 14 may determine a first amount of time between the time IMD 14 identified that patient 12 fell asleep and the time IMD 14 identified that patient 12 awoke. IMD 14 may also determine a second amount of time between the time IMD 14 identified that patient 12 began attempting to fall asleep and the time IMD 14 identified that patient 12 awoke. To calculate the sleep efficiency, IMD 14 may divide the first time by the second time.

Another sleep quality metric value that IMD 14 may calculate is sleep latency, which IMD 14 may calculate as the amount of time between the time IMD 14 identified that patient 12 was attempting to fall asleep and the time IMD 14 identified that patient 12 fell asleep. Other sleep quality metrics with values determined by IMD 14 based on the information collected by IMD 14 in the illustrated example include: total time sleeping per day, at night, and during daytime hours; number of apnea and arousal events per occurrence of sleep; and amount of time spent in the various sleep states, e.g., one or both of the S3 and S4 sleep states. IMD 14 may store the determined values as sleep quality metric values 66 within memory 48.

IMD 14 may perform the example method illustrated in FIG. 5 continuously, e.g., may monitor to identify when patient 12 is attempting to sleep and asleep any time of day, each day. In other embodiments, IMD 14 may only perform the method during evening hours and/or once every N days to conserve battery and memory resources. Further, in some embodiments, IMD 14 may only perform the method in response to receiving a command from patient 12 or a clinician via one of programmers 20, 26. For example, patient 12 may direct IMD 14 to collect sleep quality information at times when the patient believes that his or her sleep quality is low or therapy is ineffective.

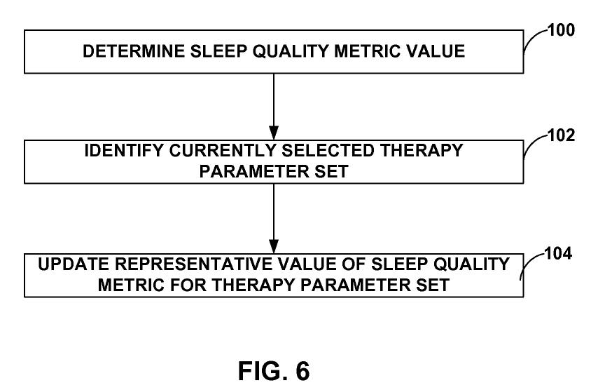

FIG. 6 is a flow diagram illustrating an example method for associating sleep quality information with therapy parameter sets 60 that may be employed by IMDs 14. An IMD 14 determines a value of a sleep quality metric according to any of the techniques described above (100). IMD 14 also identifies the current therapy parameter set, e.g., the therapy parameter set 60 used by IMD 14 to control delivery of therapy when a patient 12 was asleep (102), and associates the newly determined value with the current therapy parameter set 60.

Among sleep quality metric values 66 within memory 48, IMD 14 stores a representative value of the sleep quality metric, e.g., a mean or median value, for each of the plurality of therapy parameter sets 60. IMD 14 updates the representative values for the current therapy parameter set based on the newly determined value of the sleep quality metric. For example, a newly determined sleep efficiency value may be used to determine a new average sleep efficiency value for the current therapy parameter set 60.

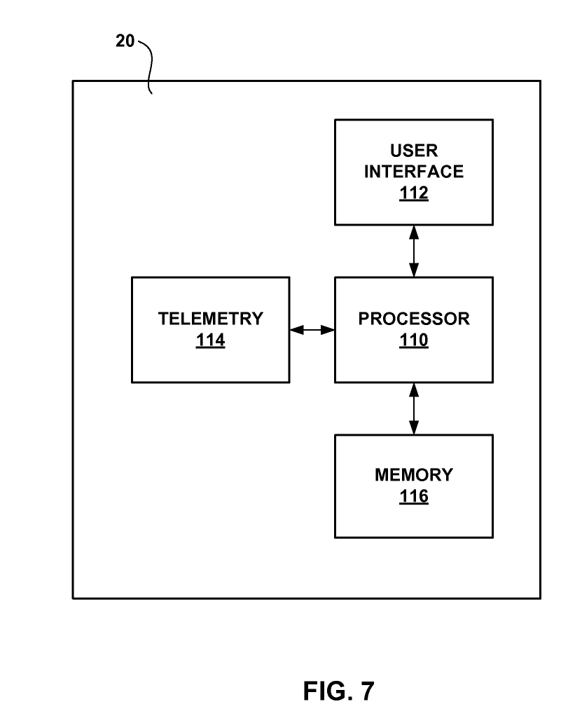

FIG. 7 is a block diagram further illustrating clinician programmer 20. A clinician may interact with a processor 110 via a user interface 112 in order to program therapy for a patient 12. Further, processor 110 may receive sleep quality metric values 66 from IMD 14 via a telemetry circuit 114, and may generate sleep quality information for presentation to the clinician via user interface 112. User interface 112 may include display 22 and keypad 24, and may also include a touch screen or peripheral pointing devices as described above. Processor 110 may include a microprocessor, a controller, a DSP, an ASIC, an FPGA, discrete logic circuitry, or the like.

Clinician programmer 20 also includes a memory 116. Memory 116 may include program instructions that, when executed by processor 110, cause clinician programmer 20 to perform the functions ascribed to clinician programmer 20 herein. Memory 116 may include any volatile, non-volatile, fixed, removable, magnetic, optical, or electrical media, such as a RAM, ROM, CD-ROM, hard disk, removable magnetic disk, memory cards or sticks, NVRAM, EEPROM, flash memory, and the like.

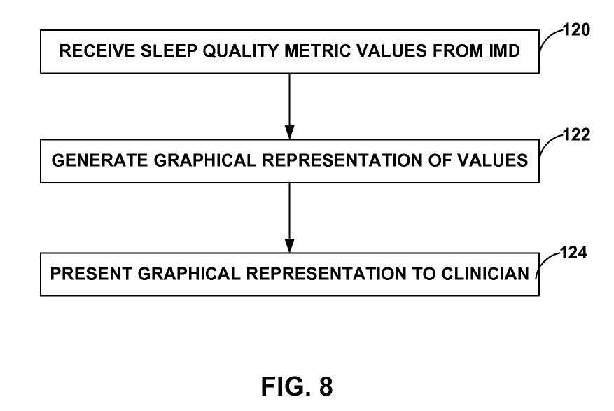

FIG. 8 is a flow diagram illustrating an example method for presenting sleep quality information to a clinician that may be employed by clinician programmer 20. Clinician programmer 20 receives sleep quality metric values 66 from an IMD 14, e.g., via telemetry circuit 114 (120). The sleep quality metric values 66 may be daily values, or mean or median values determined over greater periods of time, e.g., weeks or months.

Clinician programmer 20 may simply present the values to the clinician via display 22 in any form, such as a table of average values, or clinician programmer 20 may generate a graphical representation of the sleep quality metric values (122). For example, clinician programmer 20 may generate a trend diagram illustrating sleep quality metric values 66 over time, or a histogram, pie chart, or other graphic illustration of percentages of sleep quality metric values 66 collected by IMD 14 that were within ranges. Where clinician programmer 20 generates a graphical representation of the sleep quality metric values 66, clinician programmer 20 presents the graphical representation to the clinician via display 22 (124).

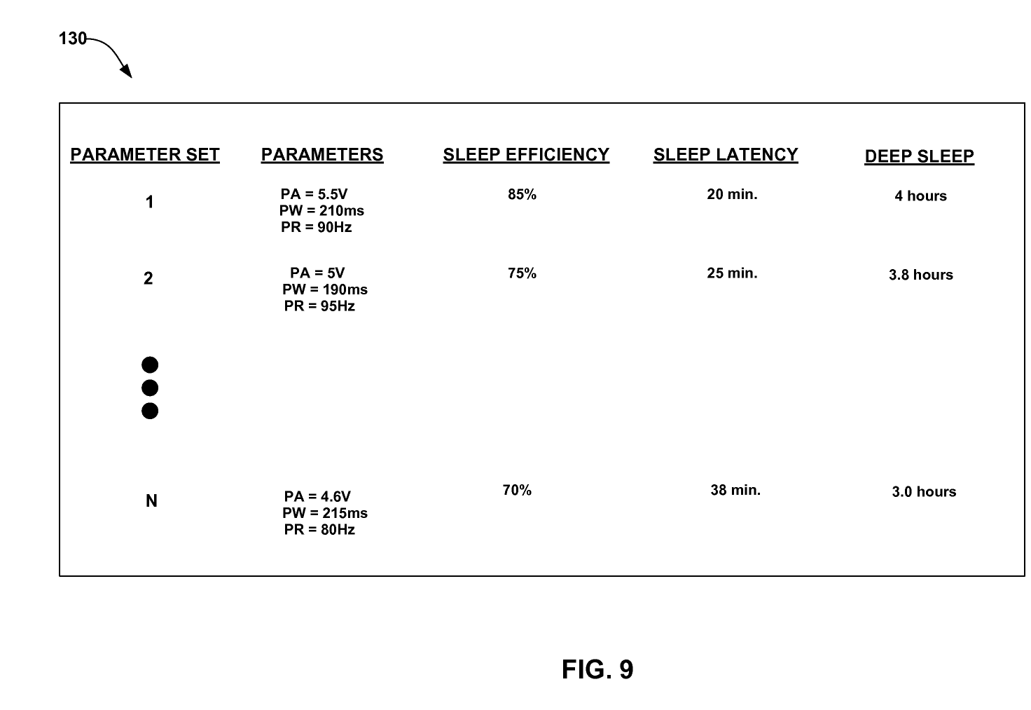

FIG. 9 illustrates an example list 130 of therapy parameter sets and associated sleep quality metric values that may be presented to a clinician by clinician programmer 20. Each row of example list 130 includes an identification of one of therapy parameter sets 60, the parameters of the set, and a representative value for one or more sleep quality metrics associated with the identified therapy parameter set, such as sleep efficiency, sleep latency, or both. The example list 130 includes representative values for sleep efficiency, sleep latency, and “deep sleep,” e.g., the average amount of time per night spent in either of the S3 and S4 sleep states.

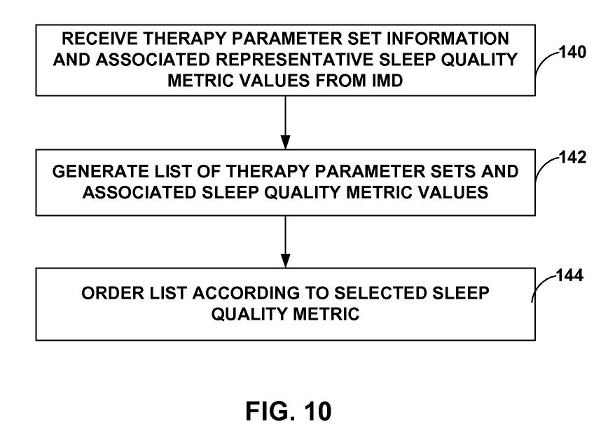

FIG. 10 is a flow diagram illustrating an example method for displaying a list 130 of therapy parameter sets and associated sleep quality information that may be employed by clinician programmer 20. According to the example method, clinician programmer 20 receives information identifying the plurality of therapy parameter sets 60 stored in memory 48 of an IMD 14, and one or more representative sleep quality metric values associated with each of the therapy parameter sets (140). Clinician programmer 20 generates a list 130 of the therapy parameter sets 60 and any associated representative sleep quality metric values (142), and orders the list according to a selected sleep quality metric (144). For example, in the example list 130 illustrated in FIG. 9, the clinician may select whether list 130 should be ordered according to sleep efficiency or sleep latency via user interface 112 of clinician programmer 20.

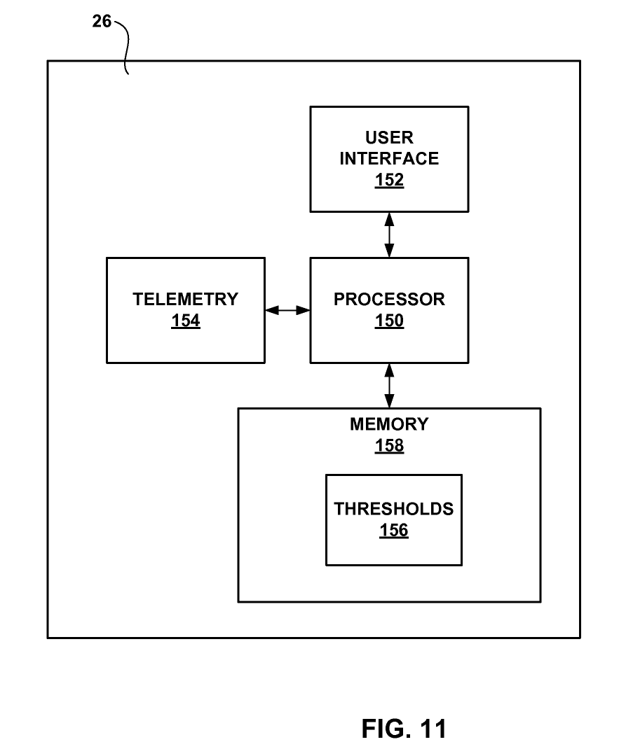

FIG. 11 is a block diagram further illustrating patient programmer 26. A patient 12 may interact with a processor 150 via a user interface 152 in order to control delivery of therapy, i.e., select or adjust one or more of therapy parameter sets 60 stored by an IMD 14. Processor 150 may also receive sleep quality metric values 66 from IMD 14 via a telemetry circuit 154, and may provide messages related to sleep quality to patient 12 via user interface 152 based on the received values. User interface 152 may include display 28 and keypad 30, and may also include a touch screen or peripheral pointing devices as described above.

In some embodiments, processor 150 may determine whether to provide a message related to sleep quality to patient 12 based on the received sleep quality metric values. For example, processor 150 may periodically receive sleep quality metric values 66 from IMD 14 when placed in telecommunicative communication with IMD 14 by patient 12, e.g., for therapy selection or adjustment. Processor 150 may compare these values to one or more thresholds 156 stored in a memory 158 to determine whether the quality of the patient's sleep is poor enough to warrant a message.

Processor 150 may present messages to patient 12 as text via display, and/or as audio via speakers included as part of user interface 152. The message may, for example, direct patient 12 to see a physician, increase therapy intensity before sleeping, or select a different therapy parameter set before sleeping than the patient had typically selected previously. In some embodiments, the message may indicate the quality of sleep to patient 12 to, for example, provide patient 12 with an objective indication of whether his or her sleep quality is good, adequate, or poor. Further, in some embodiments processor 150 may, like clinician programmer 20, receive representative sleep quality metric values. In such embodiments, processor 150 may identify a particular one or more of therapy parameter sets 60 to recommend to patient 12 based on representative sleep quality metric values associated with those programs.

Processor 150 may include a microprocessor, a controller, a DSP, an ASIC, an FPGA, discrete logic circuitry, or the like. Memory 158 may also include program instructions that, when executed by processor 150, cause patient programmer 26 to perform the functions ascribed to patient programmer 26 herein. Memory 158 may include any volatile, non-volatile, fixed, removable, magnetic, optical, or electrical media, such as a RAM, ROM, CD-ROM, hard disk, removable magnetic disk, memory cards or sticks, NVRAM, EEPROM, flash memory, and the like.

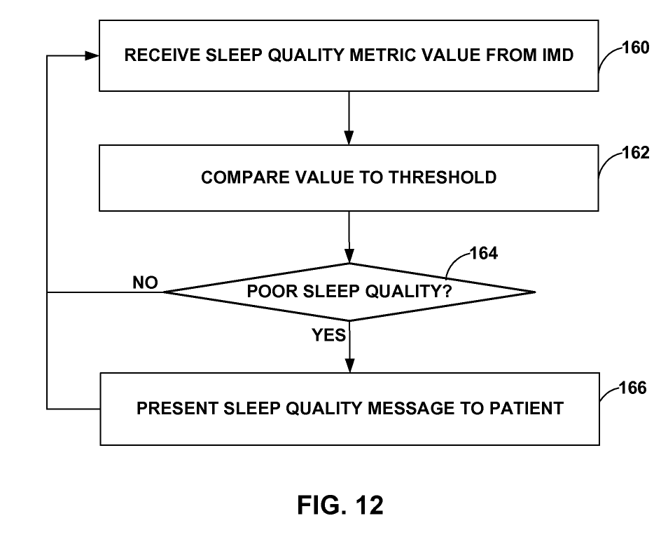

FIG. 12 is a flow diagram illustrating an example method for presenting a sleep quality message to a patient 12 that may be employed by patient programmer 26. According to the illustrated example method, patient programmer 26 receives a sleep quality metric value from IMD 14 (160), and compares the value to a threshold value 156 (162). Patient programmer 26 determines whether the comparison indicates poor sleep quality (164). If the comparison indicates that the quality of sleep experienced by patient 12 is poor, patient programmer 26 presents a message related to sleep quality to patient 12 (166).

Various embodiments of the invention have been described. However, one skilled in the art will recognize that various modifications may be made to the described embodiments without departing from the scope of the invention. For example, the invention may be embodied in any implantable medical device, including an implantable monitor that does not itself deliver a therapy to the patient. Further, the invention may be implemented via an external, e.g., non-implantable, medical device.

As discussed above, the ability of a patient to experience quality sleep, e.g., the extent to which the patient able to achieve adequate periods of undisturbed sleep in deeper, more restful sleep states, may be negatively impacted by any of a variety of ailments or symptoms. Accordingly, the sleep quality of a patient may reflect the progression, status, or severity of the ailment or symptom. Further, the sleep quality of the patient may reflect the efficacy of a particular therapy or therapy parameter set in treating the ailment or symptom. In other words, it may generally be the case that the more efficacious a therapy or therapy parameter set is, the higher quality of sleep the patient will experience.

As discussed above, in accordance with the invention, sleep quality metrics may be monitored, and used to evaluate the status, progression or severity of an ailment or symptom, or the efficacy of therapies or therapy parameter sets used to treat the ailment or symptom. As an example, chronic pain may cause a patient to have difficulty falling asleep, experience arousals during sleep, or have difficulty experiencing deeper sleep states. Systems according to the invention may monitor sleep quality metrics to evaluate the extent to which the patient is experiencing pain.

In some embodiments, systems according to the invention may include any of a variety of medical devices that deliver any of a variety of therapies to treat chronic pain, such as SCS, DBS, cranial nerve stimulation, peripheral nerve stimulation, or one or more drugs. Systems may use the techniques of the invention described above to determine sleep quality metrics for the patient and evaluate such therapies, e.g., by associating sleep quality metrics with therapy parameter sets for delivery of such therapies. Systems according to the invention may thereby evaluate the extent to which a therapy or therapy parameter set is alleviating chronic pain by evaluating the extent to which the therapy or therapy parameter set improves sleep quality for the patient.

As another example, psychological disorders may cause a patient to experience low sleep quality. Accordingly, embodiments of the invention may determine sleep quality metrics to track the status or progression of a psychological disorder, such as depression, mania, bipolar disorder, or obsessive-compulsive disorder. Further, systems according to the invention may include any of a variety of medical devices that deliver any of a variety of therapies to treat a psychological disorder, such as DBS, cranial nerve stimulation, peripheral nerve stimulation, vagal nerve stimulation, or one or more drugs. Systems may use the techniques of the invention described above to associate sleep quality metrics with the therapies or therapy parameter sets for delivery of such therapies, and thereby evaluate the extent to which a therapy or therapy parameter set is alleviating the psychological disorder by evaluating the extent to which the therapy parameter set improves the sleep quality of the patient.

Movement disorders, such as tremor, Parkinson's disease, multiple sclerosis, spasticity, and epilepsy may also affect the sleep quality experienced by a patient. The uncontrolled movements, e.g., tremor or shaking, associated such disorders, particularly in the limbs, may cause a patient to experience disturbed sleep. Accordingly, systems according to the invention may monitor sleep quality metrics to determine the state or progression of a movement disorder.

Further, systems according to the invention may include any of a variety of medical devices that deliver any of a variety of therapies to treat movement disorders, such as DBS, cortical stimulation, or one or more drugs. Baclofen, which may or may not be intrathecally delivered, is an example of a drug that may be delivered to treat movement disorders. Systems may use the techniques of the invention described above to associate sleep quality metrics with therapies or therapy parameter sets for delivery of such therapies. In this manner, such systems may allow a user to evaluate the extent to which a therapy or therapy parameter set is alleviating the movement disorder by evaluating the extent to which the therapy parameter set improves the sleep quality experienced by the patient.

Additionally, the invention is not limited to embodiments in which a programming device receives information from the medical device, or presents information to a user. Other computing devices, such as handheld computers, desktop computers, workstations, or servers. May receive information from the medical device and present information to a user as described herein with reference to programmers 20, 26. A computing device, such as a server, may receive information from the medical device and present information to a user via a network, such as a local area network (LAN), wide area network (WAN), or the Internet. In some embodiments, the medical device is an external medical device, and may itself include a display to present information to a user.

As another example, the invention may be embodied in a trial neurostimulator, which is coupled to percutaneous leads implanted within the patient to determine whether the patient is a candidate for neurostimulation, and to evaluate prospective neurostimulation therapy parameter sets. Similarly, the invention may be embodied in a trial drug pump, which is coupled to a percutaneous catheter implanted within the patient to determine whether the patient is a candidate for an implantable pump, and to evaluate prospective therapeutic agent delivery parameter sets. Sleep quality metric values collected by the trial neurostimulator or pump may be used by a clinician to evaluate the prospective therapy parameter sets, and select parameter sets for use by the later implanted non-trial neurostimulator or pump. In particular, a trial neurostimulator or pump may determine representative values of one or more sleep quality metrics for each of a plurality of prospective therapy parameter sets, and a computing device, such as a clinician programmer, may present a list of prospective parameter sets and associated representative values to a clinician. The clinician may use the list to identify potentially efficacious parameter sets, and may program a permanent implantable neurostimulator or pump for the patient with the identified parameter sets.

Further, the invention is not limited to embodiments in which an implantable or external medical device that delivers therapy to a patient determines sleep quality metric values. Instead a medical device according to the invention may record values for one or more physiological parameters, and provide the physiological parameter values to a computing device, such as one or both of programmers 20, 26. In such embodiments, the computing device, and more particularly a processor of the computing device, e.g., processors 110, 150, employs any of the techniques described herein with reference to IMD 14 in order to determine sleep quality metric values based on the physiological parameter values received from the medical device. The computing device may receive physiological parameter values from the medical device in real time, or may monitor physiological parameters of the patient by receiving and analyzing physiological parameter values recorded by the medical device over a period of time. In some embodiments, in addition to physiological parameter values, the medical device provides the computing device information identifying times at which the patient indicated that he or she was attempting to fall asleep, which the computing device may use to determine one or more sleep quality metric values as described herein.

In some embodiments, the medical device may associate recorded physiological parameter values with current therapy parameter sets. The medical device may provide information indicating the associations of recorded physiological parameter values and therapy parameter sets to the computing device, e.g., programmer 20 or 26. The computing device may determine sleep quality metric values and representative sleep quality metric values for each of the plurality of therapy parameter sets based on the physiological parameter values associated with the therapy parameter sets, as described herein with reference to IMD 14.

Additionally, the invention is not limited to embodiments in which the therapy delivering medical device monitors the physiological parameters of the patient described herein. In some embodiments, a separate monitoring device monitors values of one or more physiological parameters of the patient instead of, or in addition to, a therapy delivering medical device. The monitor may include a processor 46 and memory 48, and may be coupled to sensors 40, as illustrated above with reference to IMD 14 and FIGS. 2 and 3. The monitor may identify sleep quality metric values based on the values of the monitored physiological parameter values, or may transmit the physiological parameter values to a computing device for determination of the sleep quality metric values. In some embodiments, an external computing device, such as a programming device, may incorporate the monitor.

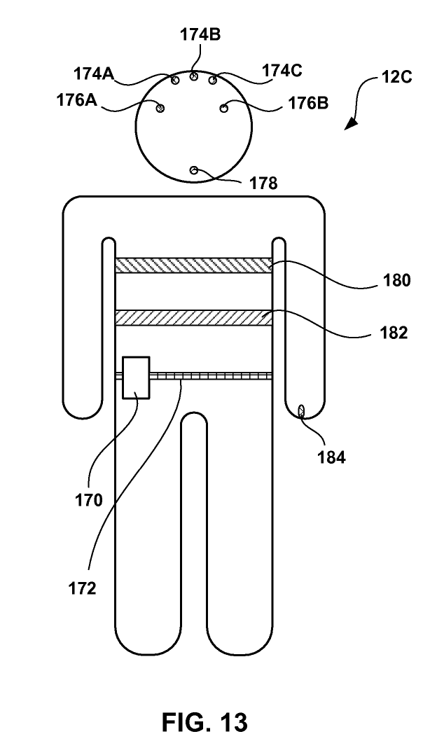

FIG. 13 is a conceptual diagram illustrating a monitor 170 that monitors values of one or more physiological parameters of the patient instead of, or in addition to, a therapy delivering medical device. In the illustrated example, monitor 170 is configured to be attached to or otherwise carried by a belt 172, and may thereby be worn by patient 12. FIG. 13 also illustrates various sensors 40 that may be coupled to monitor 170 by leads, wires, cables, or wireless connections, such as EEG electrodes 174A-C placed on the scalp of patient 12C, a plurality of EOG electrodes 176A and 176B placed proximate to the eyes of patient 12C, and one or more EMG electrodes 178 placed on the chin or jaw the patient. The number and positions of electrodes 174, 176 and 178 illustrated in FIG. 13 are merely exemplary. For example, although only three EEG electrodes 174 are illustrated in FIG. 13, an array of between 16 and 25 EEG electrodes 174 may be placed on the scalp of patient 12C, as is known in the art. EEG electrodes 174 may be individually placed on patient 12, or integrated within a cap or hair net worn by the patient. Signals received from EEG electrodes 174A-C may be analyzed to determine sleep states, e.g., using techniques and circuitry described with reference to FIG. 3.

In the illustrated example, patient 12C wears an ECG belt 180. ECG belt 180 incorporates a plurality of electrodes for sensing the electrical activity of the heart of patient 12C. The heart rate and, in some embodiments, ECG morphology of patient 12C may monitored by monitor 170 based on the signal provided by ECG belt 180. Examples of suitable belts 180 for sensing the heart rate of patient 12 are the “M” and “F” heart rate monitor models commercially available from Polar Electro. In some embodiments, instead of belt 180, patient 12C may wear a plurality of ECG electrodes attached, e.g., via adhesive patches, at various locations on the chest of the patient, as is known in the art. An ECG signal derived from the signals sensed by such an array of electrodes may enable both heart rate and ECG morphology monitoring, as is known in the art.

As shown in FIG. 13, patient 12C may also wear a respiration belt 182 that outputs a signal that varies as a function of respiration of the patient. Respiration belt 182 may be a plethysmograpy belt, and the signal output by respiration belt 182 may vary as a function of the changes is the thoracic or abdominal circumference of patient 12 that accompany breathing by the patient. An example of a suitable belt 182 is the TSD201 Respiratory Effort Transducer commercially available from Biopac Systems, Inc. Alternatively, respiration belt 182 may incorporate or be replaced by a plurality of electrodes that direct an electrical signal through the thorax of the patient, and circuitry to sense the impedance of the thorax, which varies as a function of respiration of the patient, based on the signal. In some embodiments, ECG and respiration belts 180 and 182 may be a common belt worn by patient 12, and the relative locations of belts 180 and 182 depicted in FIG. 13 are merely exemplary.

In the example illustrated by FIG. 13, patient 12C also wears a transducer 184 that outputs a signal as a function of the oxygen saturation of the blood of patient 12C. Transducer 184 may be an infrared transducer. Transducer 184 may be located on one of the fingers or earlobes of patient 12. Sensors 40 coupled to monitor 170 may additionally or alternatively include any of the variety of sensors described above that monitor any one or more of activity level, posture, heart rate, ECG morphology, respiration rate, respiratory volume, blood pressure, blood oxygen saturation, partial pressure of oxygen within blood, partial pressure of oxygen within cerebrospinal fluid, muscular activity and tone, core temperature, subcutaneous temperature, arterial blood flow, brain electrical activity, eye motion, and galvanic skin response.

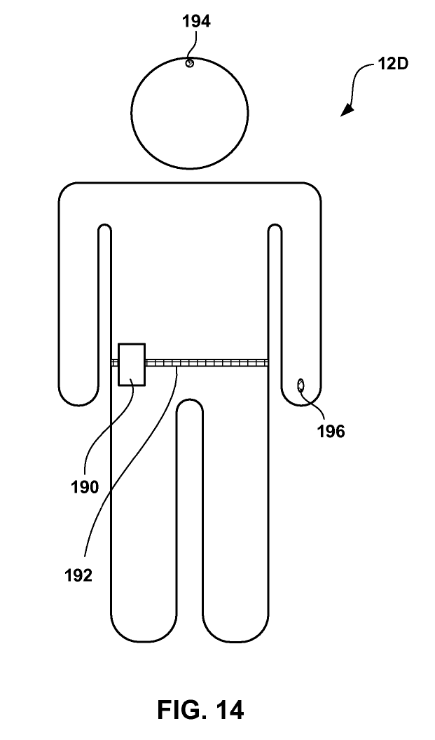

FIG. 14 is a conceptual diagram illustrating a monitor that monitors signals generated by one or more accelerometers instead of, or in addition to, monitoring of signals generated by accelerometers or other sensors by a therapy delivering medical device. As shown in FIG. 14, patient 12D is wearing monitor 190 attached to belt 192. Monitor 190 is capable of receiving measurements from one or more sensors located on or within patient 12D. In the example of FIG. 14, accelerometers 194 and 196 are attached to the head and hand of patient 12, respectively. Accelerometers 194 and 196 may measure movement of the extremities, or activity level, of patient 12 to indicate when the patient moves during sleep or at other times during the day. Alternatively, more or less accelerometers or other sensors may be used with monitor 190.

Accelerometers 194 and 196 may be preferably multi-axis accelerometers, but single-axis accelerometers may be used. As patient 12D moves, accelerometers 194 and 196 detect this movement and send the signals to monitor 190. High frequency movements of patient 12D may be indicative of tremor, Parkinson's disease, or an epileptic seizure. Accelerometers 194 and 196 may be worn externally, i.e., on a piece or clothing or a watch, or implanted at specific locations within patient 12D. In addition, accelerometers 194 and 196 may transmit signals to monitor 190 via a wireless or a wired connection.

Monitor 190 may store the measurements from accelerometers 194 and 196 in a memory. In some examples, monitor 190 may transmit the measurements from accelerometers 194 and 196 directly to another device, such as an IMD 14, programmer, or other computing device. In this case, the IMD 14, programmer, or other device may analyze the measurements from accelerometers 194 and 196 to detect efficacy of therapy, control the delivery of therapy, detect sleep or monitor sleep quality using any of the techniques described herein. In other embodiments, monitor 190 may analyze the measurements from accelerometer to detect efficacy of therapy, control the delivery of therapy, detect sleep or monitor sleep quality using any of the techniques described herein.

In some examples, a rolling window of time may be used when analyzing measurements from accelerometers 194 and 196. Absolute values determined by accelerometers 194 and 196 may drift with time or the magnitude and frequency of patient 12D movement may not be determined by a preset threshold. For this reason, it may be advantageous to normalize and analyze measurements from accelerometers 194 and 196 over a discrete window of time. For example, the rolling window may be useful in detecting epileptic seizures. If monitor 190 or an IMD 14 detects at least a predetermined number of movements over a 15 second window, an epileptic seizure may be most likely occurring. In this manner, a few quick movements from patient 12 not associated with a seizure may not trigger a response, such as recording an incident in a memory or a change in therapy.

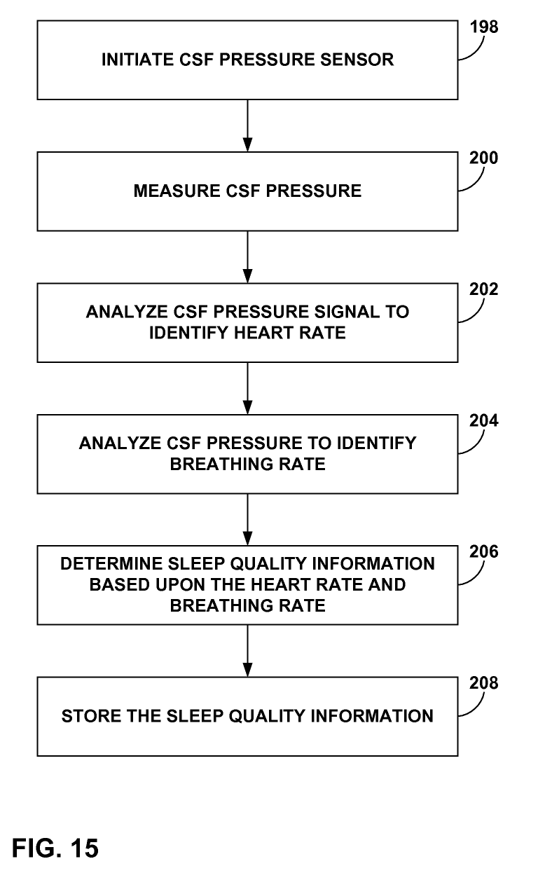

FIG. 15 is a flow diagram illustrating monitoring the heart rate and breathing rate of a patient by measuring cerebral spinal fluid pressure. As discussed above, a physiological parameter that may be measured in a patient 12 is heart rate and respiration, or breathing, rate. In the example of FIG. 15, cerebral spinal fluid (CSF) pressure may be analyzed to monitor the heart rate and breathing rate of a patient 12. A clinician initiates a CSF pressure sensor for monitoring heart rate and/or breathing rate (198). Initiating the CSF pressure sensor may include attaching a set of external electrodes or other sensors to the head of patient 12. Alternatively, the CSF pressure sensor may be implanted within the brain or spinal cord of patient 12 to acquire accurate pressure signals. The CSF pressure sensor may transfer pressure data to an implanted or external device. As an example used herein, the CSF pressure sensor transmits signal data to an IMD 14.

Once the CSF pressure sensor is initiated, the CSF pressure sensor measures CSF pressure and transmits the data to IMD 14 (200). IMD 14 analyzes the CSF pressure signal to identify the heart rate (202) and breathing rate (204) of patient 12. The heart rate and breathing rate can be identified within the overall CSF pressure signal. Higher frequency fluctuations (e.g. 40 to 150 beats per minute) can be identified as the heart rate while lower frequency fluctuations (e.g. 3 to 20 breaths per minute) in CSF pressure are the breathing rate. IMD 14 may employ filters, transformations, or other signal processing techniques to identify the heart rate and breathing rate from the CSF pressure signal.

IMD 14 may utilize the heart rate and breathing rate information when determining when patient 12 is attempting to sleep, determining when patient 12 is asleep, or otherwise determining one or more sleep quality metrics for patient 12, as described above (206). For example, faster heart rates and faster breathing rates may indicate that patient 12 is not sleeping. IMD 14 may then store values of the sleep quality metric, provide the sleep quality metric values to a programmer or other computing device, or use them to adjust stimulation therapy (208).

The invention may also be embodied as a computer-readable medium that includes instructions to cause a processor to perform any of the methods described herein. These and other embodiments are within the scope of the following claims.

1. A method comprising:
monitoring at least one physiological parameter of a patient;
determining a value of a metric that is indicative of sleep quality based on the at least one physiological parameter;
identifying a current therapy parameter used by a medical device to deliver a therapy to a patient when the value of the metric was determined, wherein the therapy comprises at least one of a movement disorder therapy, psychological disorder therapy, or deep brain stimulation; and
associating the sleep quality metric value with the current therapy parameter set.

2. The method of claim 1, wherein monitoring at least one physiological parameter comprises monitoring at least one of electrocardiogram morphology, electroencephalogram morphology, subcutaneous temperature, muscular tone, electrical activity of a brain of the patient, or eye motion.

3. The method of claim 1, wherein monitoring at least one physiological parameter comprises monitoring at least one of activity level, posture, heart rate, respiration rate, respiratory volume, or core temperature.

4. The method of claim 1, wherein monitoring at least one physiological parameter comprises monitoring at least one of blood pressure, blood oxygen saturation, partial pressure of oxygen within blood, partial pressure of oxygen within cerebrospinal fluid, muscular activity, arterial blood flow, melatonin level within a bodily fluid, or galvanic skin response.

5. The method of claim 1, wherein the sleep quality metric comprises sleep efficiency, and determining the value of the sleep quality metric comprises:
identifying when the patient is attempting to sleep;
identifying when the patient is asleep; and
determining a percentage of time that the patient is asleep while the patient is attempting to sleep.

6. The method of claim 1, wherein the sleep quality metric comprises sleep latency, and determining the value of the sleep quality metric comprises:
identifying a first time when the patient is attempting to fall asleep;
identifying a second time when the patient falls asleep; and
determining an amount of time between the first and second times.

7. The method of claim 1, wherein determining the value of the sleep quality metric comprises:
identifying when the patient is within a sleep state; and
determining an amount of time that the patient was within the sleep state, wherein the sleep state comprises at least one of an S3 sleep state and an S4 sleep state.

8. The method of claim 1, wherein determining a value of the sleep quality metric comprises:
determining a value of each of a plurality of sleep quality metrics; and
determining a value of an overall sleep quality metric based on the plurality of sleep quality metric values.

9. The method of claim 8, wherein determining a value of an overall sleep quality metric comprises applying a weighting factor to at least one of the plurality of sleep quality metric values.

10. The method of claim 1,
wherein monitoring at least one physiological parameter of a patient comprises monitoring a frequency of an electroencephalogram (EEG) signal, and
wherein determining a value of the metric that is indicative of sleep quality comprises identifying a sleep state based on the frequency of the EEG signal and determining the value of the metric based on the sleep state.

11. The method of claim 1, further comprising:
determining a plurality of values of the sleep quality metric over time; and
presenting sleep quality information to a user based on the plurality of values.

12. The method of claim 11, wherein presenting sleep quality information to the user comprises presenting at least one of a trend diagram, a histogram, or a pie chart based on the plurality of values of the sleep quality metric.

13. The method of claim 11, wherein presenting sleep quality information to a user comprises presenting a message related to sleep quality to the patient via a patient programmer.

14. The method of claim 1, further comprising:
determining a plurality of values of the sleep quality metric over time;
associating each of the determined values of the sleep quality metric with a current therapy parameter set according to which therapy was delivered by the sleep quality metric value was determined; and
for each of a plurality of therapy parameter sets, determining a representative value of the sleep quality metric based on the values of the sleep quality metric associated with the therapy parameter set.

15. The method of claim 14, further comprising:
presenting a list of the therapy parameter sets and the associated representative values to a user; and
ordering the list of therapy parameter sets according to the associated representative values.

16. The method of claim 14, further comprising:
determining a plurality of values over time for each of a plurality of metrics that are indicative of sleep quality;
associating each of the determined values with a current therapy parameter set according to which therapy was delivered by the sleep quality metric value was determined; and
for each of the therapy parameter sets, determining a representative value for each of the sleep quality metrics based on the values of that sleep quality metric associated with the therapy parameter set.

17. The method of claim 16, further comprising:
presenting a list of the therapy parameter sets and the associated representative values to a user; and
ordering the list of therapy parameter sets according to the representative values of a user-selected one of the sleep quality metrics.

18. A medical system comprising:
a medical device that delivers at least one of a movement disorder therapy, psychological disorder therapy, or deep brain stimulation to a patient;
a monitor that monitors at least one physiological parameter of the patient based on a signal received from at least one sensor; and
a processor that determines a value of a metric that is indicative of sleep quality based on the at least one physiological parameter, identifies a current therapy parameter set used by the medical device to deliver the therapy to the patient when the value of the sleep quality metric was determined, and associates the sleep quality metric value with the current therapy parameter set.

19. The medical system of claim 18, wherein the processor:
identifies when the patient is attempting to sleep;
identifies when the patient is asleep; and
determines a percentage of time that the patient is asleep while the patient is attempting to sleep to determine the value of the sleep quality metric.

20. The medical system of claim 18, wherein the processor:
identifies when the patient is asleep; and
identifies at least one of a number of arousal events and a number of apnea events during a period of sleep to determine the value of the sleep quality metric.

21. The medical system of claim 18, wherein the processor:
identifies when the patient is within a sleep state; and
determines an amount of time that the patient was within the sleep state to determine the value of the sleep quality metric, wherein the sleep state comprises at least one of an S3 sleep state or an S4 sleep state.

22. The medical system of claim 18, further comprising a module that monitors an electroencephalogram (EEG) signal and determines a sleep state according to a frequency of the EEG signal, wherein the processor determines the value of the metric that is indicative of sleep quality based on the sleep state.

23. The medical system of claim 18, further comprising a user interface that presents sleep quality information to a user based upon a plurality of values of the sleep quality metric.

24. The medical system of claim 18, wherein the processor:
determines a plurality of values of the sleep quality metric over time;
associates each of the determined values of the sleep quality metric with a current therapy parameter set according to which therapy was delivered by the sleep quality metric value was determined; and
for each of a plurality of therapy parameter sets, determines a representative value of the sleep quality metric based on the values of the sleep quality metric associated with the therapy parameter set.

25. The medical system of claim 24, further comprising a user interface that presents a list of the therapy parameter sets and the associated representative values to a user, wherein the processor orders the list of therapy parameter sets according to the associated representative values.

26. The medical system of claim 24, wherein the processor:
determines a plurality of values over time for each of a plurality of metrics that are indicative of sleep quality;
associates each of the determined values with a current therapy parameter set according to which therapy was delivered by the sleep quality metric value was determined; and
for each of the therapy parameter sets, determines a representative value for each of the sleep quality metrics based on the values of that sleep quality metric associated with the therapy parameter set.

27. The medical system of claim 26, further comprising a user interface that presents a list of the therapy parameter sets and the associated representative values to a user, wherein the processor orders the list of therapy parameter sets according to the representative values of a user-selected one of the sleep quality metrics.

28. A computer-readable medium comprising instructions that cause a processor to:
monitor at least one physiological parameter of a patient;
determine a value of a metric that is indicative of sleep quality based on the at least one physiological parameter;
identify a current therapy parameter used by a medical device to deliver a therapy to a patient when the value of the metric was determined, wherein the therapy comprises at least one of a movement disorder therapy, psychological disorder therapy, or deep brain stimulation; and
associate the sleep quality metric value with the current therapy parameter set.

29. The computer-readable medium of claim 28, further comprising instructions that cause the processor to:
determine a plurality of values of the sleep quality metric over time;
associate each of the determined values of the sleep quality metric with a current therapy parameter set according to which therapy was delivered by the sleep quality metric value was determined; and
for each of a plurality of therapy parameter sets, determine a representative value of the sleep quality metric based on the values of the sleep quality metric associated with the therapy parameter set.

30. The computer-readable medium of claim 29, further comprising instructions that cause the processor to:
present a list of the therapy parameter sets and the associated representative values to a user; and
order the list of therapy parameter sets according to the associated representative values.

31. The computer-readable medium of claim 29, further comprising instructions that cause the processor to:
determine a plurality of values over time for each of a plurality of metrics that are indicative of sleep quality;
associate each of the determined values with a current therapy parameter set according to which therapy was delivered by the sleep quality metric value was determined; and
for each of the therapy parameter sets, determine a representative value for each of the sleep quality metrics based on the values of that sleep quality metric associated with the therapy parameter set.

32. The computer-readable medium of claim 31, further comprising instructions that cause the processor to:
present a list of the therapy parameter sets and the associated representative values to a user; and
order the list of therapy parameter sets according to the representative values of a user-selected one of the sleep quality metrics.
